(19) United States
(12) Patent Application Publication
(10) Pub. No.: US 2025/0248395 A1
(43) Pub. Date: Aug. 7, 2025

(54) ANTIMICROBIAL POROUS FRAMEWORKS

(71) Applicant: THE UNIVERSITY OF BRISTOL, Bristol (GB)

(72) Inventor: Benjamin Charles BAKER, Birmingham West Midlands (GB)

(21) Appl. No.: 18/855,492

(22) PCT Filed: Apr. 12, 2023
(86) PCT No.: PCT/GB2023/050985

§ 371 Date: Oct. 9, 2024

(30) Foreign Application Priority Data
Apr. 13, 2022 (GB) 2205496.9

(51) Int. Cl. A01N 25/10; A01N 43/78; A01P 1/00; A61L 15/44; A61L 27/54; A61L 31/16

(52) U.S. Cl. CPC A01N 25/10; A01N 43/78; A01P 1/00; A61L 15/44; A61L 27/54; A61L 31/16

(57) ABSTRACT

A framework comprising hub units joined by linker units, wherein: a) the shortest path from the most central atom of each hub unit to the most central atom of an adjacent hub unit, inclusive of the most central atoms, is on average 19 atoms or more, and/or b) the framework is bonded to an antimicrobial agent, and/or c) the framework is complexed with an antimicrobial agent.

### FIELD OF THE INVENTION

The present invention relates to antimicrobial frameworks, in particular antibacterial frameworks. The frameworks comprise hub units joined by linker units, wherein: a) the shortest path from the most central atom of each hub unit to the most central atom of an adjacent hub unit, inclusive of the most central atoms, is on average 19 atoms or more, and/or b) the framework is bonded to an antimicrobial agent, and/or c) the framework is complexed with an antimicrobial agent. Such frameworks can be used to form articles that have inherent antimicrobial activity.

### BACKGROUND OF THE INVENTION

The requirement for articles to be kept free of infectious microbes, such as bacteria, is consistently growing in importance.

Bacterial resistance has become an increasingly large problem in recent decades, as the efficacy of small molecule antibiotics are challenged by new strains of bacteria that have evolved to evade them.

Bacterial infections may be caused by exposure to an article with bacteria on its surface. Articles made of certain materials may exacerbate bacterial infections.

Bacterial infections may be particularly troublesome in areas such as healthcare settings, for example for medical devices (e.g. implants such as catheters, or wound dressings).

For example, many items of PPE are fabricated from passive structural materials to provide a physical barrier to pathogens. PPE may be either single-use, where it should be disposed of after it is used, or reusable. However, users may reuse PPE that is designed to be single-use in order to reduce the environmental impact of the PPE and/or to reduce the cost and inconvenience of regularly purchasing PPE.

It is troubling that PPE, such as facemasks and visors, can be contaminated with microbes, such as bacteria, either from before use, where they have not been packaged in a sterile manner, or where they are going to be reused.

The presence of microbes on the PPE can present a health risk to the user despite their best intentions to reduce their health risk by using the PPE to reduce or eliminate their exposure to pathogens.

It will be appreciated that antibacterial surfaces will also find applications in agriculture, air treatment, water treatment, food preparation, and surface coatings (e.g. paint).

The present invention has been devised with the foregoing in mind.

### SUMMARY OF THE INVENTION

According to a first aspect, the present invention provides a framework comprising hub units joined by linker units, wherein:
each hub unit comprises an X group bonded to three or more R¹ groups, wherein:
   each X group independently represents N, P or a C1-10 hydrocarbon group that is optionally substituted;
   each R¹ group independently represents a C3-10 (hetero)aryl group that is optionally substituted;
each linker unit links two or more hub units together and is represented by the formula -L-(R²-Lₒ)ₙ-, wherein:
   each L group is independently selected from the list consisting of: —CH₂—, —(CH₂)₂—, —(CH)₂—, —CC—, —OCH₂—, —SCH₂—, —P═CH—, —PHCH₂—, —N═CH—, —NHCH₂—, —O—, —O₂—, —OS—, —S—, —SO₂—, —S(O)—, —S₂—, —N═N—, —ONH—, —NH—, —P═N—, —P(O)HNH—, —POOHNH—, —OPOOHNH—, —P(O)HO—, —POOHO—, —OPOOHO—, —P═P—, —OPH—, —PH—, —C(O)—, —C(O)O—, —C(O)NH—, —N(C(O)—)₂, —NHC(O)NH—, —NHC(O)O—, —NHS(O)₂—;
   each R² group independently represents a C3-20 (hetero)aryl group that is optionally substituted; and
   each n is 1 or more; and
the R¹ groups of the hub unit are bonded to L groups of the linker units, such that three or more linker units are bonded to each hub unit; and
   a) the shortest path from the most central atom of each hub unit to the most central atom of an adjacent hub unit, inclusive of the most central atoms, is on average 19 atoms or more, and/or
   b) the framework is bonded to an antimicrobial agent, and/or
   c) the framework is complexed with an antimicrobial agent.

According to a second aspect the claimed invention provides a use of a framework of the first aspect as an antimicrobial (e.g. antibacterial) agent.

The present inventors have surprisingly determined that frameworks, as defined by the first aspect, exhibit activity against microbes. In particular, the polymers as defined by the first aspect exhibit antibacterial activity, for example against Moraxella catarrhalis (MX, gram negative) and Staphylococcus aureus (SA, gram positive). It is also surprising that frameworks as defined by the first aspect can be active against both gram-positive and gram-negative bacteria.

Firstly, it has been determined that the distance between adjacent hub units is particularly important for the antimicrobial (e.g. antibacterial) activity of the framework. The present invention thus defines that the shortest path from the most central atom of each hub unit to the most central atom of an adjacent hub unit, inclusive of the most central atoms, is 19 atoms or more on average. The length of the shortest path may be determined by diffusion NMR.

Secondly, it has been determined that the framework can be bonded to an antimicrobial agent. Surprisingly, when the framework is bonded to an antimicrobial agent, the antimicrobial activity can be retained.

Thirdly, it has been determined that the framework exhibits antimicrobial (e.g. antibacterial) activity when it is complexed with an antimicrobial agent, such as methylene blue, chromium(III) (for example a salt or oxide thereof, e.g. Cr₂O₃), and/or amoxycillin.

It has surprisingly been determined that the framework holds an absorbed antimicrobial agent, even after filtration and Soxhlet extraction (and even with several solvents of different polarities).

Furthermore, it is surprising that the antimicrobial agent retains its activity even when it is complexed with the framework, and even after filtration and Soxhlet extraction.

Therefore, the framework containing the antimicrobial agent can robustly include antimicrobial agents within useful materials. The antimicrobial effects of frameworks with antimicrobial agents absorbed therein are very robust. Furthermore, this illustrates that a wide variety of manufacturing techniques may be used to prepare the frameworks, whilst still retaining the antimicrobial activity. Such techniques may be used, for example, to purify the framework so that it meets certain technical specifications.

It is considered that the framework will not exhibit photocatalytic activity as the antibacterial activity of the frameworks was observed following incubation in the dark. Thus, the antimicrobial activity of the framework is thought to be independent of any photocatalytic activity. This enables the framework to achieve antimicrobial activity even in the absence of light (e.g. blue or UV light), for example indoors and/or while in storage before use or in between uses of an article comprising the framework.

It will be appreciated that the frameworks of the present invention may be called polymers. The frameworks of the present invention can be used to prepare articles.

The prior art does not disclose that any frameworks, themselves, have antibacterial properties. Further, there is little guidance with regard to the protocols by which the antibacterial properties of such materials could be tested, due to their scarcity.

CN111202060A discloses organic frameworks such as TPA-DMTP (tris(4-aminophenyl)amine-2,5-dimethoxybenzene-1,4-dicarbaldehyde), TPB-DMTP (1,3,5-tris(4-nitrophenyl)benzene-2,5-dimethoxybenzene-1,4-dicarbaldehyde) and TPT-DMTP (2,4,6-tris(4-aminophenyl)-1,3,5-triazine-2,5-dimethoxybenzene-1,4-dicarbaldehyde) frameworks are complexed with antibacterial molecules.

L. Bai et al., Chem. Commun., 2016, 52, 4128-4131 discloses covalent organic frameworks complexed with drugs.

J. Chen et al., Agnew. Chem. Int. Ed., 2019, 58, 11715-11719 discloses poly(triphenylamine), PTPA.

However, none of these documents disclose that the frameworks of the present invention by themselves would have antibacterial properties.

Importantly, the frameworks of the present invention have been found to display antibacterial properties themselves when the shortest path from the most central atom of each hub unit to the most central atom of an adjacent hub unit, inclusive of the most central atoms, is on average 19 atoms or more.

It is not necessary to complex such frameworks of the invention with other materials that have antibacterial properties, or to absorb such materials into the frameworks, in order for the frameworks to have antibacterial properties.

It has also been found that the frameworks are antimicrobial when they are bonded to or complexed with an antimicrobial agent.

Surprisingly, the antimicrobial properties of the frameworks of the present invention persist when the framework is blended with another material to form an article.

It has been determined that the frameworks of the invention are not cytotoxic. Therefore, the frameworks can be used for articles that come into contact with living tissues, such as implants.

According to a third aspect, the present invention provides an article comprising a framework as defined by the first aspect.

The articles will have an inherent and active antimicrobial activity because they comprise the framework of the first aspect.

The articles may be articles of personal protective equipment (PPE). Such articles of PPE will have an inherent and active antimicrobial activity.

As such, microbes that have been introduced to an article, such as an article of PPE, prior to use can be eliminated prior to use. Furthermore, microbes that have been introduced to an article, such as an article of PPE, during use can be eliminated during use or prior to subsequent use.

Therefore, the present invention provides significant benefits in terms of reducing the health risk presented to the user of an article, such as an item of PPE.

It will be appreciated that articles having antimicrobial, such as antibacterial, activity are sought-after in a number of other fields, including other healthcare-related fields, water processing, air treatment/handling (e.g. ventilation) and food and drink manufacture.

The framework may suitably be included in a composition along with a carrier, for example to enable three-dimensional (3D) printing of the framework. Thus, according to a fourth aspect, the present invention provides a composition comprising a framework of the first aspect and a carrier. The composition may be a 3D-printable ink. The carrier may include or be an organic solvent and/or a polymer.

The present disclosure includes the subject-matter of the following clauses:

1. A framework comprising hub units joined by linker units, wherein: each hub unit comprises an X group bonded to three or more R¹ groups, wherein: each X group independently represents N, P or a C1-10 hydrocarbon group that is optionally substituted; each R¹ group independently represents a C3-10 (hetero)aryl group that is optionally substituted; each linker unit links two or more hub units together and is represented by the formula -L-(R²-Lₒ)ₙ-, wherein: each L group is independently selected from the list consisting of: —CH₂—, —(CH₂)₂—, —(CH)₂—, —CC—, —OCH₂—, —SCH₂—, —P═CH—, —PHCH₂—, —N═CH—, —NHCH₂—, —O—, —O₂—, —OS—, —S—, —SO₂—, —S(O)—, —S₂—, —N═N—, —ONH—, —NH—, —P═N—, —P(O)HNH—, —POOHNH—, —OPOOHNH—, —P(O)HO—, —POOHO—, —OPOOHO—, —P═P—, —OPH—, —PH—, —C(O)—, —C(O)O—, —C(O)NH—, —N(C(O)—)₂, —NHC(O)NH—, —NHC(O)O—, —NHS(O)₂—; each R² group independently represents a C3-20 (hetero)aryl group that is optionally substituted; each o is 1 or more; and each n is 1 or more; and the R¹ groups of the hub unit are bonded to L groups of the linker units, such that three or more linker units are bonded to each hub unit; and a) the shortest path from the most central atom of each hub unit to the most central atom of an adjacent hub unit, inclusive of the most central atoms, is on average 19 atoms or more, and/or b) the framework is bonded to an antimicrobial agent, and/or c) the framework is complexed with an antimicrobial agent.

2. The framework of clause 1, wherein each hub unit comprises an X group bonded to three R¹ groups.

3. The framework of clause 1 or clause 2, wherein each X group independently represents N or a C3-8 (hetero)aryl group.

4. The framework of any preceding clause, wherein each R¹ group independently represents a C3-10 aryl group.

5. The framework of any preceding clause, wherein each L independently represents a group selected from the list consisting of: —OCH₂—, —SCH₂—, —N═CH—, —NHCH₂—, —O—, —S—, —NH—, —C(O)O—, —C(O)NH—, —N(C(O)—)₂, —NHC(O)NH—, and —NHC(O)O—.

6. The framework of any preceding clause, wherein each R² independently represents a C3-18 aryl group that is optionally substituted.

7. The framework of any preceding clause, wherein the shortest path from the most central atom of each hub unit to the most central atom of an adjacent hub unit, inclusive of the most central atoms, is on average 24 or more atoms.

8. The framework of clause 7, wherein the shortest path is on average from 24 to 400 atoms.

9. The framework of any preceding clause, wherein 90% or more of the linker units are attached to two or more hub units.

10. The framework of any preceding clause, wherein 80% or more of the hub units are attached to three or more linker units.

11. The framework of any preceding clause, wherein n is 2 or more.

12. The framework of any preceding clause, wherein the antimicrobial agent is an antiviral agent, an antifungal agent and/or an antibacterial agent.

13. An article comprising a framework as defined by any preceding clause.

14. The article of clause 13, wherein the article is an article of personal protective equipment.

15. The article of clause 14, wherein the article is a face mask.

16. The article of any one of clauses 13 to 15, wherein the framework forms at least the part of the article comprising the surface, or wherein the framework is included in a coating on the surface of the article.

17. A composition comprising a framework of any one of clauses 1 to 12 and a carrier.

18. The composition of clause 17, wherein the carrier includes an organic solvent.

19. The composition of clause 17 or clause 18, wherein the carrier includes a polymer.

### DETAILED DESCRIPTION OF THE INVENTION

The term “alkyl” refers to linear, branched, and cyclic (i.e. cycloalkyl) saturated hydrocarbon groups. Examples of such groups include methyl, ethyl, n-propyl, isopropyl, n-butyl, isobutyl, sec-butyl, tert-butyl, n-pentyl, isopentyl, neopentyl or hexyl, cyclopropyl, cyclobutyl, cyclopentyl, and cyclohexyl.

The term “alkenyl” as used herein refers to a linear or branched hydrocarbon group containing one or more carbon-carbon double bond. Examples of such groups include vinyl, allyl, prenyl, and isoprenyl.

The term “(hetero)aryl” encompasses aryl and/or heteroaryl groups.

The term “aryl” as used herein refers to carbocyclic aromatic groups including phenyl, naphthyl, indenyl, and tetrahydronaphthyl groups. Aryl groups can be monocyclic or polycyclic (e.g. bicyclic), as long as at least one ring is aromatic. Aryl groups can be a five membered or six membered monocyclic ring or a bicyclic structure, for example formed from fused five and six membered rings or two or more (e.g. five) fused five or six membered rings, such as perylene.

The term “heteroaryl” as used herein refers to aromatic ring systems that contain heteroatoms (e.g. N, O, S) within the ring structure. Heteroaryl groups can be monocyclic or polycyclic (e.g. bicyclic), as long as at least one ring is aromatic. Heteroaryl groups can be a five membered or six membered monocyclic ring or a bicyclic structure formed from fused five and six membered rings or two fused six membered rings. Each ring may contain up to five heteroatoms typically selected from N, S and O. Typically the heteroaryl ring will contain up to 4 heteroatoms, more typically up to 3 heteroatoms, more usually up to 2, for example a single heteroatom, e.g. from 1 to 3 heteroatoms. In one embodiment, the heteroaryl ring contains at least one ring nitrogen atom.

Examples of heteroaryl groups include pyrrole, furan, thiophene, imidazole, furazan, oxazole, oxadiazole, oxatriazole, isoxazole, thiazole, thiadiazole, isothiazole, pyrazole, triazole and tetrazole, pyridine, pyrazine, pyridazine, pyrimidine and triazine groups.

Examples of monocyclic groups are groups containing 4, 5, 6, 7 and 8 ring members (i.e. ring atoms), more usually 4 to 7, and preferably 5, 6 or 7 ring members, more preferably 5 or 6 ring members. Examples of bicyclic groups are those containing 8, 9 and 10 ring members.

The term “halogen” as used herein refers to a fluorine, chlorine, bromine or iodine atom. In one embodiment, the halogen group is fluorine, chlorine or bromine. In another embodiment, it is chlorine or bromine.

The denomination “C[number]” in relation to a group defines the number of carbon atoms in the group. The denomination “C[number 1]-[number 2]” in relation to a group defines that the number of carbon atoms in the group ranges from [number 1] to [number 2]. For example, “C6-30 alkyl” represents an alkyl group containing from 6 to 30 carbon atoms.

### Framework

The framework of the claimed invention comprises hub units joined by linker units, wherein:
each hub unit comprises an X group bonded to three or more R¹ groups,
each linker unit links two or more hub units and is represented by the formula -L-(R²-Lₒ)ₙ-, and
the R¹ groups of the hub unit are bonded to the L groups of the linker units, such that three or more linker units are bonded to each hub unit.

The frameworks may not be poly(triphenylamine). The frameworks may not be TPA-DMTP, TPB-DMTP and/or TPT-DMTP.

The specific requirements of the groups are defined in more detail below. The framework of the present invention may be termed a polymer. The structure of the framework therefore extends in three or more directions from the hubs, through linker units and to other hub units.

Each linker unit links two or more hub units together. Where a linker unit links three hub units together, o is 2 and each linker unit is represented by the formula -L-(R²-Lₒ)ₙ-, and it will be understood that all three linker groups can link to hub units. Preferably the linker units links two hub units together, o is 1 and each linker unit is represented by the formula -L-(R²-Lₒ)ₙ-. Preferably o is from 1 to 3, such as 1 or 2.

The framework of the present invention may comprise the following unit structure:

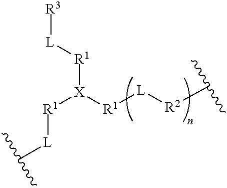

It will be understood that the unit structure of the framework repeats. The unit structure joins with other unit structures at the locations shown by the wavy lines such that, in the framework, the or each R² group of this unit structure is attached to two L groups.

In other words, the wavy lines signify positions where the unit structure joins to other unit structures. The unit structure joins with other unit structures such that the or each R² group of this unit structure attaches to an L group of another unit structure, and such that the terminal L group of this unit structure attaches to the R² group of another unit structure. For example, the following structure represents two of the unit structures linked together:

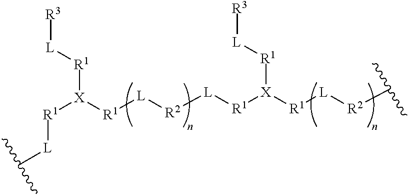

The framework can naturally have termini, where the repetition of the unit structure ceases. For example, this may be where a hub unit is not attached to a linker unit, or where a linker unit is not attached to a hub unit. At the termini, for example, the wavy lines may be substituted for H or another atom depending on the starting materials, reagents and/or conditions used to prepare the framework.

In the framework, it may be that 90% or more, such as 95% or more or 98% or more, or 99% or more, by moles, of the linker units are attached to two or more hub (preferably two) units. It may be that 80% or more, such as 90% or more or 96% or more, or 98% or more, by moles, of the hub units are attached to three or more (e.g. three) linker units. For example, it may be that 90% or more, by moles, of the linker units are attached to two or more (preferably two) hub units and that 80% or more by moles, of the hub units are attached to three or more (e.g. three) linker units.

The framework including complexed antimicrobial (e.g. antibacterial) agent may be made using a process comprising the steps of:
providing a solution, e.g. an aqueous solution, of the antimicrobial agent, and
contacting the solution with the framework.

The framework including complexed antimicrobial (e.g. antibacterial) agent may be purified or otherwise treated by subjecting the framework to filtration and/or Soxhlet extraction, for example Soxhlet extraction in water.

Each hub unit may be termed “trivalent” or at least trivalent, as they normally bond to three or more linker units. Preferably each hub unit has a C₃ symmetry element. Each linker unit may be termed “divalent” or at least divalent, as they normally bond to two or more hub units. Preferably each hub unit is trivalent and normally bonds to three linker units. Preferably each linker unit is divalent and normally bonds to two hub units. More preferably each hub unit is trivalent and normally bonds to three linker units, and each linker unit is divalent and normally bonds to two hub units.

It has been determined that the distance between adjacent hub units is particularly important for the antimicrobial activity of the framework. In this scenario, antimicrobial activity is observed even without an antimicrobial agent being bonded to or complexed with the framework. This may be because the pore size of the framework is controlled by the distance between adjacent hub units. It is postulated that this allows microbes to interact more closely with the pores of the framework, and therefore to experience the antimicrobial effects of the framework and/or antimicrobial agent bonded thereto. Antimicrobial activity is observed when the distance between adjacent hub units of frameworks is increased, i.e. when the pores in the framework are larger.

The distance between adjacent hub units of frameworks may be defined by i) the average (e.g. mean) shortest path from the most central atom of a hub unit (i.e. the most central atom of an X group) in the framework to the most central atom of an adjacent hub unit (i.e. the most central atom of the X group of an adjacent hub unit, inclusive of the most central atoms), and/or ii) the average (e.g. mean) shortest path from one end of a linker unit (where the linker unit bonds to one hub unit) to another end of the linker unit (where the linker unit bonds to another hub unit). The framework of the claimed invention requires definition (i), but definition (ii) is a suitable alternative. The average is a number average.

It will be understood that the shortest path is the shortest route that can be made through covalent bonds between two defined points. The length of the shortest path may be determined by diffusion nuclear magnetic resonance (NMR) spectroscopy.

The average (e.g. mean) shortest path from one end of a linker unit (where the linker unit bonds to one hub unit) to the other/another end of the linker unit (where the linker unit bonds to another hub unit) may be 9 or more, or 10 or more atoms in length, for example 11 or more atoms, or 12 or more atoms in length. Preferably the average shortest path between the ends is 13 or more, such as 14 or more, such as 15 or more atoms in length. The average shortest path between the ends may be from 9 to 500 atoms in length, such as from 9 to 400 atoms, or from 10 to 300 atoms, for example from 10 to 250 atoms. The average shortest path between the ends may be from 12 to 500 atoms in length, such as from 12 to 400 atoms, or from 12 to 300 atoms, for example from 12 to 250 atoms, or from 12 to 100 atoms.

The average shortest path from the most central atom of a hub unit (i.e. the most central atom of an X group) in the framework to the most central atom of an adjacent hub unit (i.e. the most central atom of the X group of an adjacent hub unit, inclusive of the most central atoms) may be 19 or more, or 20 or more atoms in length, for example 24 or more atoms, or 28 or more atoms in length. Preferably the average shortest path is 32 or more, such as 36 or more atoms in length. The average shortest path may be from 19 to 500 atoms in length, such as from 19 to 400 atoms, or from 20 to 300 atoms, for example from 20 to 250 atoms. The average shortest path may be from 24 to 500 atoms in length, such as from 24 to 400 atoms, or from 24 to 300 atoms, for example from 24 to 250 atoms, or from 24 to 100 atoms.

Where X is, for example, a hydrocarbon (e.g. aryl or heteroaryl ring), there may be more than one atom that is the “most central” atom, but the shortest path should be determined from the atom that is closest to the centre of the hub unit and that defines the shortest path to the adjacent hub unit. The most central atom of the hub unit is that furthest (in terms of atoms) from the linker units.

It will be understood that the value for n and the shortest path from the most central atom of each hub unit to the most central atom of an adjacent hub unit, inclusive of the most central atoms, are directly related. As n increases, so does the length of the shortest path.

The framework may be defined in terms of its method of synthesis instead of or as well as the requirements for n and the length of the shortest path. For example, the framework may be prepared by a method comprising controlling the molar ratios of reagents to control the linker length, such as for n to be 1 or more (or other values as discussed above), and/or to control the shortest path from the most central atom of each hub unit to the most central atom of an adjacent hub unit, inclusive of the most central atoms, to be on average 19 atoms or more (or other values as discussed above). Increasing the molar ratios of reagents for linker units compared to the hub units would, for example, increase the value of n and of the shortest chain length. The molar ratio of reagents for hub units to reagents for linker units may be controlled to be 1:3 or more, preferably 1:4 or more, or 1:5 or more, such as 1:8 or more, or 1:10 or more, or 1:20 or more. The molar ratio of reagents for hub units to reagents for linker units may be from 1:3 to 1:1000, such as from 1:3 to 1:500, or from 1:3 to 1:200, for example from 1:3 to 1:50. Three or more times more linker unit reagent is normally used compared to the hub unit reagent since each hub unit is bonded to three or more linker units.

Thus, the present invention may define a framework comprising hub units joined by linker units, wherein: each hub unit comprises an X group bonded to three or more R¹ groups, wherein: each X group independently represents N, P or a C1-10 hydrocarbon group that is optionally substituted; each R¹ group independently represents a C3-10 (hetero)aryl group that is optionally substituted; and each linker unit links two or more hub units together and is represented by the formula -L-(R²-Lₒ)ₙ-, wherein: each L group is independently selected from the list consisting of: —CH₂—, —(CH₂)₂—, —(CH)₂—, —CC—, —OCH₂—, —SCH₂—, —P═CH—, —PHCH₂—, —N═CH—, —NHCH₂—, —O—, —O₂—, —OS—, —S—, —SO₂—, —S(O)—, —S₂—, —N═N—, —ONH—, —NH—, —P═N—, —P(O)HNH—, —POOHNH—, —OPOOHNH—, —P(O)HO—, —POOHO—, —OPOOHO—, —P═P—, —OPH—, —PH—, —C(O)—, —C(O)O—, —C(O)NH—, —N(C(O)—)₂, —NHC(O)NH—, —NHC(O)O—, —NHS(O)₂—; each R² group independently represents a C3-20 (hetero)aryl group that is optionally substituted; and each n is 1 or more; and the R¹ groups of the hub unit are bonded to L groups of the linker units, such that three or more linker units are bonded to each hub unit; and a) the framework is obtainable by a method comprising controlling the molar ratios of reagents to control the linker length, preferably to control the shortest path from the most central atom of each hub unit to the most central atom of an adjacent hub unit, inclusive of the most central atoms, to be on average 19 atoms or more, and/or b) the framework is bonded to an antimicrobial agent, and/or c) the framework is complexed with an antimicrobial agent.

### X

Each hub unit includes an X group. The X group bonds to three or more R¹ groups and may be termed “trivalent” or “more than trivalent”. The X group may bond to three or four R¹ groups. Preferably the X group bonds to three R¹ groups and is therefore termed “trivalent”.

Each X group independently represents N, P or a C1-10 hydrocarbon group that is optionally substituted. Each X group may be substituted by one or more Y group. Each X group may independently represent N, P, C1-3 alkyl (e.g. C1-2 alkyl, preferably C1 alkyl) or a C3-10 (hetero)aryl group that is optionally substituted, for example by one or more Y group. For example, X may represent N or a C3-10 (hetero)aryl group. X may represent N or a C3-8 (hetero)aryl group (e.g. a C3-6 (hetero)aryl group). The C3-10 (hetero)aryl group may be a C3-10 heteroaryl group. For example, the C3-10 (hetero)aryl group may be selected from the list consisting of: phenyl, pyrrole, furan, thiophene, furazan, oxazole, oxadiazole, oxatriazole, isoxazole, thiazole, thiadiazole, isothiazole, pyrazole, triazole, imidazole, indole, pyrrole, quinoline, isoquinoline, pyridine, pyrimidine, triazine (e.g. 1,3,5-triazine), pyridazine and pyrazine. Preferably, the C3-10 heteroaryl group is selected from the list consisting of: pyridine, pyrimidine, triazine (e.g. 1,3,5-triazine), pyridazine and pyrazine.

The (hetero)aryl group may include from 5 to 7 ring atoms, for example 5 or 6 ring atoms, preferably 6 ring atoms.

The C3-10 heteroaryl group may include from 1 to 5 heteroatoms, for example wherein the heteroatoms are selected from the list consisting of N, P, O and S. The number of heteroatoms may be from 2 to 4, most preferably 3. Preferably the C3-10 heteroaryl group includes from 1 to 5 heteroatoms selected from the list of N and O. More preferably the C3-10 heteroaryl group includes from 2 to 4, e.g. 3, heteroatoms, e.g. wherein the heteroatoms are N.

Preferably the X group has a C₃ symmetry element.

### R¹

Each R¹ group independently represents a C3-10 (hetero)aryl group that is optionally substituted, for example with one or more Y group. Preferably each R¹ group is not substituted.

Each R¹ group may independently represent a C3-8 (hetero)aryl group, for example a C3-7 (hetero)aryl group, or a C3-6 (hetero)aryl group. Each R¹ may independently represent a C4-8 (hetero)aryl group, for example a C4-7 (hetero)aryl group, or a C4-6 (hetero)aryl group. Preferably each R¹ independently represents a C5-8 (hetero)aryl group, for example a C5-7 (hetero)aryl group, or a C5-6 (hetero)aryl group.

Preferably each R¹ group independently represents a C3-10 aryl group, such as a C3-8 aryl group, for example a C3-7 aryl group, or a C3-6 aryl group. Each R¹ may independently represent a C4-8 aryl group, for example a C4-7 aryl group, or a C4-6 aryl group. Preferably each R¹ independently represents a C5-8 aryl group, for example a C5-7 aryl group, or a C5-6 aryl group.

Preferably R¹ includes a 6-membered (hetero)aryl ring, for example a phenyl group. Preferably the 6-membered (hetero)aryl ring is para-substituted, for example a para-substituted phenyl group (i.e. where the L and X groups are para to one another).

Preferably all R¹ groups are the same, but they may be different.

### L

Each L independently represents a group selected from the list consisting of: —CH₂—, —(CH₂)₂—, —(CH)₂—, —CC—, —OCH₂—, —SCH₂—, —P═CH—, —PHCH₂—, —N═CH—, —NHCH₂—, —O—, —O₂—, —OS—, —S—, —SO₂—, —S(O)—, —S₂—, —N═N—, —ONH—, —NH—, —P═N—, —P(O)HNH—, —POOHNH—, —OPOOHNH—, —P(O)HO—, —POOHO—, —OPOOHO—, —P═P—, —OPH—, —PH—, —C(O)—, —C(O)O—, —C(O)NH—, —N(C(O)—)₂, —NHC(O)NH—, —NHC(O)O—, and —NHS(O)₂—.

Each L may independently represent a group selected from the list consisting of: —CH₂—, —(CH₂)₂—, —OCH₂—, —SCH₂—, —N═CH—, —NHCH₂—, —O—, —S—, —SO₂—, —S(O)—, —NH—, —C(O)—, —C(O)O—, —C(O)NH—, —N(C(O)—)₂, —NHC(O)NH—, —NHC(O)O—, and —NHS(O)₂—. For example, each L may independently represent a group selected from the list consisting of: —CH₂—, —(CH₂)₂—, —OCH₂—, —SCH₂—, —N═CH—, —NHCH₂—, —O—, —S—, —NH—, —C(O)—, —C(O)O—, —C(O)NH—, —N(C(O)—)₂, —NHC(O)NH— and —NHC(O)O—. Each L may independently represent a group selected from the list consisting of: —OCH₂—, —SCH₂—, —N═CH—, —NHCH₂—, —O—, —S—, —SO₂—, —S(O)—, —NH—, —C(O)—, —C(O)O—, —C(O)NH—, —N(C(O)—)₂, —NHC(O)NH—, —NHC(O)O—, and —NHS(O)₂—. Each L may independently represent a group selected from the list consisting of: —OCH₂—, —SCH₂—, —N═CH—, —NHCH₂—, —O—, —S—, —NH—, —C(O)O—, —C(O)NH—, —N(C(O)—)₂, —NHC(O)NH—, and —NHC(O)O—.

Preferably each L independently represents a group selected from the list consisting of: —OCH₂—, —SCH₂—, —N═CH—, —NHCH₂—, —O—, —S—, —NH—, —C(O)NH—, —N(C(O)—)₂, and —NHC(O)NH—. More preferably each L independently represents a group selected from the list consisting of: —SCH₂—, —N═CH—, —NHCH₂—, —S—, —NH—, and —N(C(O)—)₂. Most preferably each L independently represents a group selected from the list consisting of: —N═CH—, —S—, —NH—, and —N(C(O)—)₂.

The L groups in the framework may all be the same or the framework may include two or more different L groups. Preferably all L groups in the framework are the same.

### R²

Each R² group independently represents a C3-20 (hetero)aryl group that is optionally substituted, for example by one or more Y group.

Each R² may independently represent a C3-18 (hetero)aryl group, such as a C3-16 (hetero)aryl group, or a C3-14 (hetero)aryl group that is optionally substituted. Preferably, each R² independently represents a C3-12 (hetero)aryl group, such as a C5-12 (hetero)aryl group, for example a C6-12 (hetero)aryl group that is optionally substituted. More preferably, each R² independently represents a C3-10 (hetero)aryl group, such as a C5-10 (hetero)aryl group, for example a C6-10 (hetero)aryl group that is optionally substituted.

Preferably each R² is an aryl group. Each R² may independently represent a C3-18 aryl group, such as a C3-16 aryl group, or a C3-14 aryl group. Preferably, each R² independently represents a C3-12 aryl group, such as a C5-12 aryl group, for example a C6-12 aryl group. More preferably, each R² independently represents a C3-10 aryl group, such as a C5-10 aryl group, for example a C6-10 aryl group.

For example, each R² may independently be selected from phenyl or napthyl. Preferably each R² is independently be selected from 1,4-substituted phenyl or 1,4,5,8-substituted napthyl.

The R² groups in the framework may all be the same or the framework may include two or more different R² groups. Preferably all R² groups in the framework are the same.

### Substituents

Each X, R¹ and R² group is optionally substituted.

Each X, R¹ and R² group may be substituted with one or more internal substituent and/or one or more external substituent. The term “internal substituent” refers to substituents provided between two carbon atoms, i.e. within a hydrocarbon group. The term “external substituent” refers to substituents provided in the replacement of a hydrogen atom of a C—H bond, i.e., on the edge of a hydrocarbon group. Preferably any substituents on the X and/or R¹ groups are external substituents.

Internal substituents can include where one or more (e.g., two or more) of the carbon atoms in the hydrocarbon chain are replaced with heteroatoms. The heteroatom(s) may, for example, be selected from O, N, S, SO, SO₂, P, B, Si, and combinations thereof. For example, the heteroatom(s) may be selected from O, N, S, and combinations thereof. In one embodiment from 1 to 5 carbon atoms in the group are replaced with heteroatom(s), e.g., 1, 2 or 3 carbon atoms in the group might be replaced with heteroatom(s). When more than one carbon atom in the group is replaced, the heteroatoms used may be the same or may be different.

Therefore, for example, the X, R¹ and/or R² group may include an ether, amine, imine, thioether, sulfoxide, sulfone, and/or sulfonamide group. Amine and imine substituents are particularly preferred. The number of carbon atoms in the group may be reduced where one or more of the carbon atoms in the group are replaced with heteroatoms. However, the skilled person would readily be able to see how many carbon atoms would have been in the group had one or more of these not been replaced with heteroatoms.

In the X, R¹ and/or R² group, one or more (e.g. two or more) of the hydrogen atoms of the group may be replaced with substituent groups. In one embodiment from 1 to 10 hydrogen atoms in the group are substituted, such as from 1 to 6, e.g. 1, 2, 3 or 4 of the hydrogen atoms in the group might be replaced with substituent groups. When more than one hydrogen atom in the X, R¹ and/or R² group is replaced, the substituent groups used may be the same or may be different. For example, the X, R¹ and/or R² group may be substituted with one or more substituent groups independently selected from hydroxyl and amino and carboxyl groups, and aryl or heteroaryl groups (especially unsaturated cyclic and heterocyclic groups with 5 to 10 atoms (e.g. 6 to 10 atoms) in their ring, such as imidazolyl, thiazolyl, thienyl, phenyl, tolyl, xylyl, pyridinyl, pyrimidinyl, pyrazinyl, indolyl or naphthyl groups). The group may be substituted with one or more substituent groups independently selected from hydroxyl and amino and carboxyl groups. Carboxylic acid groups are particularly preferred, especially where a cationic antimicrobial agent (e.g. a metal cation, e.g. chromium(III), or methylene blue) is to be complexed with the framework. The total number of carbon atoms in each of the substituent groups may be from 0 to 12, such as from 0 to 6 or from 1 to 4.

The or each substituent may independently be selected from the list consisting of: —CH₂—, —(CH₂)₂—, —(CH)₂—, —CC—, —OCH₂—, —SCH₂—, —P═CH—, —PHCH₂—, —N═CH—, —NHCH₂—, —O—, —O₂—, —OS—, —S—, —SO₂—, —S(O)—, —S₂—, —N═N—, —ONH—, —NH—, —P═N—, —P(O)HNH—, —POOHNH—, —OPOOHNH—, —P(O)HO—, —POOHO—, —OPOOHO—, —P═P—, —OPH—, —PH—, —C(O)—, —C(O)O—, —C(O)NH—, —N(C(O)—)₂, —NHC(O)NH—, —NHC(O)O—, and —NHS(O)₂—. These represent internal substituents.

Each X group and each R² group may optionally be substituted by one or more Y group independently selected from the list consisting of: cyano, halogen (e.g. F, Cl, Br and I), N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —OP(O)₂OR^{Z}, —S(O)₂NHR^{Z}, —NHS(O)₂R^{Z}, —NR^{Z}₂, —NHR^{Z} and —OR^{Z}. These represent external substituents. For example, the Y groups may be selected from the list consisting of: cyano, halogen (e.g. F, Cl, Br and I), N₃, —C(O)R^{Z}, —C(O)OR^{Z}, —OC(O)R^{Z}, —C(O)NHR^{Z}, —NHC(O)R^{Z}, —NR^{Z}₂, —NHR^{Z} and —OR^{Z}. The Y groups may be selected from the list consisting of: halogen (e.g. F and Cl), —C(O)OR^{Z}, —OC(O)R^{Z}, —C(O)NHR^{Z}, —NHC(O)R^{Z} and —OR^{Z}.

Any R^{Z} group independently represents H, C1-8 alkyl, C2-8 alkenyl, C6-8 aryl, or C4-8 heterocyclyl. Each R^{Z} may independently represent H, C1-6 alkyl, C2-6 alkenyl, C6 aryl, or C4-6 heterocyclyl. For example, each R^{Z} may independently represent H, C1-4 alkyl, or C2-4 alkenyl.

X and/or R¹ may be substituted with 5 or fewer Y groups, such as 3 or fewer, or 2 or fewer Y groups. Preferably the X and/or R¹ groups is/are not substituted with any Y groups.

Preferably, Y groups substituting R² are selected from the list consisting of: —C(O)OR^{Z}, —OC(O)R^{Z}, —C(O)NHR^{Z}, —NHC(O)R^{Z} and —OR^{Z}, especially where R^{Z} represents H, C1-4 alkyl, or C2-4 alkenyl. Preferably, Y groups substituting R² are selected from the list consisting of: —C(O)OH, —C(O)NH₂, and —OH. More preferably, R² is substituted with one or more Y group that is —C(O)OH.

R² may be substituted with 5 or fewer Y groups, such as 3 or fewer, or 2 or fewer Y groups. Preferably R² is optionally substituted with from 1 to 3 Y groups, for example from 1 to 2, most preferably 1 optional Y group. For example, R² may be substituted with from 0 to 3 Y groups selected from the list consisting of: —C(O)OR^{Z}, —OC(O)R^{Z}, —C(O)NHR^{Z}, —NHC(O)R^{Z} and —OR^{Z}, where R^{Z} independently represents H, C1-4 alkyl, or C2-4 alkenyl. Preferably R² is optionally substituted with 1 Y group that is selected from the list consisting of: —C(O)OH, —C(O)NH₂, and —OH. More preferably R² is optionally substituted with 1 Y group that is —C(O)OH.

Any substituents chosen may be designed to facilitate coordination with an antimicrobial agent. For example, a substituent that is or can be anionic, e.g. aprotic, (for example an acidic substituent, such as a carboxylic acid) may be chosen to facilitate coordination with a cationic antimicrobial agent (such as methylene blue).

n and m

n is 1 or more, merely signifying that there is at least one R² group and at least two L groups in each linker unit. It may be that n is 2 or more and optionally the framework is complexed with an antimicrobial (e.g. antibacterial) agent.

In one embodiment n is 2 or more and the framework is not complexed with an antimicrobial (e.g. antibacterial) agent. In one embodiment n is 1 or more and the framework includes one or more antimicrobial (e.g. antibacterial) agent bonded to the hub unit, wherein the bond between the antimicrobial agent and the hub unit is direct or via an -L-(R²-Lₒ)ₘ- group, wherein m is 0 or more. In one embodiment n is 1 or more and the framework is complexed with an antimicrobial agent.

n may be 3 or more, such as 5 or more, or 7 or more, or 10 or more, such as 15 or more, or 30 or more, for example 35 or more. n may be 200 or lower, such as 100 or lower, or 60 or lower, such as 50 or lower. n may be 45 or lower, such as 30 or lower, or 15 or lower, such as 12 or lower. n may be from 2 to 200, such as from 2 to 100 or from 2 to 50. n may be from 3 to 200, such as from 3 to 100 or from 3 to 50.

It is postulated that larger pore sizes are provided by n being larger, and that this allows microbes to more closely interact with the pores to experience the antimicrobial effects of the framework and/or antimicrobial agent bonded thereto.

The antimicrobial agent may be bonded to each hub unit and/or each linker unit. The bond between the antimicrobial agent and the hub unit may be direct or via an -L-(R²-Lₒ)ₘ- group, wherein m is 0 or more.

Each m may be 1 or more or 2 or more, such as 3 or more or 5 or more, or 7 or more, or 10 or more, such as 15 or more, or 30 or more, for example 35 or more. Each m may be 200 or lower, such as 100 or lower, or 60 or lower, such as 50 or lower. Each m may be 45 or lower, such as 30 or lower, or 15 or lower, such as 12 or lower. Each m may be from 0 to 200, such as from 2 to 100 or from 2 to 50. Each m may be from 3 to 200, such as from 3 to 100 or from 3 to 50.

Preferably each n and/or m may be treated as an average (e.g. a number average), for example a mean average, of the number of repeat units.

### R³

R³ is shown in some structures relating to embodiments of the present invention. R³ represents either:
—R²- [symbol] , wherein the resulting -LR²- group repeats n times, or
—R² group bonded to an antimicrobial agent, or
an antimicrobial agent.

Preferably R³ represents either:
—R²- [symbol] , wherein the resulting -LR²- group repeats n times, or
—R² group bonded to an antimicrobial agent.

Where R³ represents R²- [symbol] , and the resulting -LR²- group repeats n times, the unit structure can be shown as:

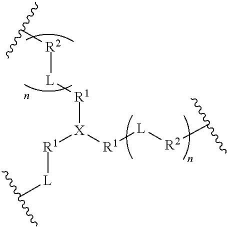

L, R² and n are discussed in more detail above.

### Antimicrobial Agents

The framework may be bonded to and/or complexed with an antimicrobial agent.

The antimicrobial agent may be an antiviral agent, an antifungal agent and/or an antibacterial agent. Preferably the antimicrobial agent is an antibacterial agent.

The framework complexed with an antimicrobial (e.g. antibacterial) agent may be defined as having the antimicrobial agent absorbed therein.

The antimicrobial agent itself is preferably water-soluble, especially where the framework is complexed with the antimicrobial agent. However, it will be understood that this does not mean that the antimicrobial agent can leach from the framework.

The complex of the framework and the antimicrobial agent may be formed due to hydrogen bonding and/or pi-pi stacking. Thus, preferably, the antimicrobial agent is a hydrogen bond donor, hydrogen bond acceptor, and/or includes a (hetero)aromatic group.

f

The antimicrobial (e.g. antibacterial) agent may be an organic molecule or an inorganic salt.

Examples of antimicrobial (e.g. antibacterial) agents that are organic molecules include those that are suitable for administration to mammals (e.g. humans) and/or other animals, such as medicaments, and those that are not suitable for such administration.

Specific examples of antibacterial agents that are organic molecules include triclosan, triclocarban, chlorhexidine, iodine, phenols e.g. polyphenols, carbolic acid and cresylic acid, benzalkonium chloride, benzethonium chloride, chloroxylenol, β-lactams, penicillins, cephalosporins, ampicillin, amoxicillin, amoxicillin/clavulanic acid (co-amoxiclav), β-Lactamase-sensitive, first generation including penicillin G, benzathine penicillin G, penicillin V, procaine penicillin, propicillin, pheneticillin, azidocillin, clometocillin, and penamecillin, quinolones such as flouroquinolone, Maxaquin (lomefloxacin), Floxin (ofloxacin), Noroxin (norfloxacin), Tequin (gatifloxacin), Cipro (ciprofloxacin), Avelox (moxifloxacin), Levaquin (levofloxacin), Factive (gemifloxacin), Cinobac (cinoxacin), NegGram (nalidixic acid), Trovan (trovafloxacin), and Zagam (sparfloxacin), clavulanate, latamoxef, loracarbef, oxacephem, carbacephems, cephalothin, cephaloridine, cephazolin, β-lactamase-resistant, 1st generation e.g. cloxacillin (dicloxacillin flucloxacillin), methicillin, nafcillin, oxacillin and temocillin, aminoglycosides including kanamycin A, B and C, amikacin, tobramycin, dibekacin, gentamicin, sisomicin, netilmicin, neomycins B, C and neomycin E (paromomycin), streptomycin, neomycin, toframycin, cephalosporins (first generation and second generation), cephalosporins (third, fourth, and fifth generations), vancomycin, clindamycin, isoniazid, rifampin, ethambutol, pyrazinamide, bacitracin, polymixins, sulfonamides, glycopeptide and nitroimidazoles, carbepenems (e.g. imipenems), macrolides such as erythromycin, roxithromycin, clarithromycin, azithromycin, and dirithromycin, tetracyclines e.g. tetracycline, chlortetracycline, oxytetracycline, demeclocycline, lymecycline, meclocycline, methacycline, minocycline, and tigecycline, chloramphenicol, ticarcillin (a carboxypenicillin), rifamycins, Streptogramins e.g. dalfopristin and quinopristin, sulphonamides, coumarins, flavonoids, naphthoquinones such as lapachol, plumbagone, juglone and lawsone, alkaloids, organosulphur compounds such as thiosulfinates and glucosinolates, iridoids, secoiridoids, saponins, terpenoids such as Curcumin, gingerols, cineole, citral, geraniol, linalool and menthol, terpinolene, terpineol and terpineolene, limonoids such as swietenolide and 2-hydroxy-3-O-tigloylswietenolide, polyacetylenes such as falcarinol, falcarindiol, panaxydiol, 8-O-methylfalcarindiol, methyl deca-4,6-diynoate, deca-4,6-diynoic acid, deca-4,6-diyne, dimethyl octa-3,5-diyne-1,8-dioate, deca-4,6-diyne-1,10-dioic acid, deca-4,6-diynoic acid, deca-4,6-diyne-1,10-dioic acid, 13(E),17-octadecadiene-9,11-diynoic acid, 17-octadecene-9,11,13-triynoic acid, and pentayne diol, anthranoids, Crystal violet, Undecylenic acid and methylene blue.

The antimicrobial agent may be a chaotropic agent, such as urea and/or guanidine.

Examples of antimicrobial agents include salts, such as salts of organic acids, such as acetic acid, lactic acid or citric acid, such as the sodium salts of these acids. These are examples of antibacterial agents.

Antimicrobial agents include metals, oxides thereof, salts thereof and complexes thereof, for example mercury, arsenic, copper, silver, zinc, antimony, aluminium, bismuth, chromium (e.g. chromium(III)) and nickel, and oxides, salts and complexes thereof. These are examples of antibacterial agents.

Preferably the antimicrobial agent is selected from the list of methylene blue, chromium(III) (for example a salt or oxide thereof, e.g. Cr₂O₃), and/or amoxycillin. These are examples of antibacterial agents.

In one embodiment the antimicrobial (e.g. antibacterial) agent is a salt, wherein the ion of the antimicrobial agent that provides the antimicrobial effect is cationic.

Examples of antiviral agents include those selected from the list consisting of: Abacavir, Acyclovir (Aciclovir), Adefovir, Amantadine, Ampligen, Amprenavir (Agenerase), Umifenovir (Arbidol), Atazanavir, Atripla, Baloxavir marboxil (Xofluza), Biktarvy, Boceprevir, Bulevirtide, Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Edoxudine, Efavirenz, Elvitegravir, Emtricitabine, Enfuvirtide, Entecavir, Etravirine (Intelence), Famciclovir, Fomivirsen, Fosamprenavir, Foscarnet, Ganciclovir (Cytovene), Ibacitabine, Ibalizumab (Trogarzo), Idoxuridine, Imiquimod, Imunovir, Indinavir, Lamivudine, Letermovir (Prevymis), Lopinavir, Loviride, Maraviroc, Methisazone, Moroxydine, Nelfinavir, Nevirapine, Nexavir formerly (Kutapressin), Nitazoxanide, Norvir, Oseltamivir (Tamiflu), Penciclovir, Peramivir, Penciclovir, Peramivir (Rapivab), Pleconaril, Podophyllotoxin, Raltegravir, Remdesivir, Ribavirin, Rilpivirine (Edurant), Rilpivirine, Rimantadine, Ritonavir, Saquinavir, Simeprevir (Olysio), Sofosbuvir, Stavudine, Taribavirin (Viramidine), Telaprevir, Telbivudine (Tyzeka), Tenofovir alafenamide, Tenofovir disoproxil, Tipranavir, Trifluridine, Trizivir, Tromantadine, Truvada, Umifenovir, Valaciclovir (Valtrex), Valganciclovir (Valcyte), Vicriviroc, Vidarabine, Zalcitabine, Zanamivir (Relenza), and Zidovudine.

Preferred examples of antiviral agents include those selected from the list consisting of: zanamivir, peramivir and baloxavir marboxil. Baloxavir marboxil is particularly preferred due to the sterically accessible aromatic groups that it contains.

Examples of antifungal agents include those selected from the list consisting of: Mancozeb, Myclobutanil, Copper, Sulfur, Phosphorous acid, mycoviruses, α-Cadinol, Citronella oil, Gmelinol, Hinokitiol, Jojoba oil, Mesquitol, Nimbin, Polyene antifungals such as Amphotericin B, Candicidin, Filipin, Hamycin, Natamycin, Nystatin and Rimocidin, azole antifungals such as Imidazoles (e.g. Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, and Tioconazole), Triazoles (e.g. Albaconazole, Efinaconazole, Epoxiconazole, Fluconazole, Isavuconazole, Itraconazole, Posaconazole, Propiconazole, Ravuconazole, Terconazole, and Voriconazole), and Thiazoles (e.g. Abafungin), allylamines (e.g. butenafine, naftifine, and terbinafine), Echinocandins (e.g. Anidulafungin, Caspofungin, and Micafungin), Triterpenoids (e.g. Ibrexafungerp), Acrisorcin, Amorolfine, Aurones, Benzoic acid, Carbol fuchsin, Ciclopirox (ciclopirox olamine), Clioquinol, Coal tar, Copper(II) salts (e.g. Copper(II) sulfate), Crystal violet, chlorophetanol, Diiodohydroxyquinoline (Iodoquinol), Flucytosine (5-fluorocytosine), Fumagillin, Griseofulvin, Haloprogin, Miltefosine, Nikkomycin, Orotomide (F901318), Piroctone olamine, Pentanenitrile, Potassium iodide, Selenium disulfide, Sodium thiosulfate, Thiocarbamates (e.g. Tolnaftate), Triacetin, Undecylenic acid and Zinc pyrithione.

The antimicrobial agent may be held within the framework. It has been found that antimicrobial agents present in an aqueous solution can be absorbed into the framework and are retained by the framework, even after filtration and Soxhlet extraction.

The antimicrobial agent may be bonded to the framework. As such, the antimicrobial agent may contain a functional group suitable for bonding with the framework. Preferably, the antimicrobial agent contains an amine (e.g. a primary amine) that the framework can bond to. The antimicrobial agent may be an agent selected from the lists above that contains an amine group, such as a primary amine group. This is particularly preferred where the framework includes a COOH group, for example as a substituent of the linker unit (e.g. the R² group).

The framework may include the (bonded and/or complexed) antimicrobial agent in an amount of 0.001 wt % or more, relative to the total weight of the framework, such as 0.01 wt % or more, or 0.05 wt % or more, preferably 0.1 wt % or more, such as 0.5 wt % or more. The amount of antimicrobial agent in the framework may be 50 wt % or less, such as 20 wt % or less, or 10 wt % or less, for example 5 wt % or less. The amount of antimicrobial agent in the framework may be from 0.001 to 50 wt %, such as from 0.05 wt % to 20 wt %.

### Articles

It has been found that the antibacterial properties of the frameworks of the present invention persist when the framework is blended with another material to form an article.

The framework of the present invention may be used to form an article. The article comprises a framework of the present invention.

The framework may be used to form all of the article, or the framework may be used to form at least the part of the article comprising the surface, or the framework may be used to coat the surface of the article. The article may be formed from a composite comprising the framework and another material (e.g. a structural material), such as a plastics material. The plastics material may, for example, be silicone (e.g. silicone rubber) and/or polyurethane.

The framework may be particularly useful as a surface coating on frequently touched surfaces, such as a handrail, a door, a door handle, a door push plate, a hospital bed, a wheelchair.

The article may be an article of personal protective equipment (PPE), such as a face mask (e.g. a surgical mask or a respirator mask), a face visor, a shield, an apron, a gown, a glove, a pair of goggles or safety glasses. These articles are particularly useful in healthcare settings, where the prevention of transmission or propagation of microbes, such as bacteria, is particularly desirable. In one embodiment, the article is a component in a medical environment; preferably the article is a component in a hospital or a veterinary hospital.

The article may be another article where the prevention of transmission or propagation of microbes, such as bacteria, is particularly desirable, for example as a medical device such as a surgical instrument, or an implant or prosthesis, or a medical machine or component thereof.

In particular, the article may be selected from surgical instruments, such as forceps, reamers, pushers, pliers, or retractors; permanent implants, such as artificial heart valves, voice prostheses, prosthetic joints, implanted artificial lenses, stents (e.g. vascular stents), and shunts (e.g. hydrocephalus shunts); and non-permanent implants, such as pacemakers and pacemaker leads, drain tubes, endotracheal or gastrointestinal tubes, temporary or trial prosthetic joints, surgical pins, guidewires, surgical staples, cannulas, subcutaneous or transcutaneous ports, and indwelling catheters and catheter connectors, and contact lenses. The article may also be a medical machine or component thereof, for example, it may be selected from dialysis machines, dialysis water delivery systems, water circuits within a dialysis unit and water delivery systems for respirator therapy. The article may be a dressing (e.g. a wound dressing).

In one embodiment the article is a catheter. Examples of indwelling catheters include urinary catheters, vascular catheters (e.g., central venous catheters, dialysis catheters, peripheral venous catheters, arterial catheters and pulmonary artery Swan-Ganz catheters), peritoneal dialysis catheters, central venous catheters and needleless connectors.

In one embodiment the article is a dressing, for example a hydrocolloid dressing, a hydrogel dressing, an alginate dressing, a collagen dressing, a foam dressing, a transparent dressing, a cloth dressing, gauze (e.g. paraffin gauze), a low adherent dressing, or a semipermeable film (e.g. polyurethane coated with acrylic) dressing.

The present invention allows the prevention of medical device-associated bacterial infections by providing the framework that is antibacterial. For example, the framework of the present invention offers the possibility to effectively reduce catheter-related bacterial infections.

It is also contemplated that the frameworks are useful for coating onto food preparation surfaces, such as kitchen counters, cutting boards, sinks, stoves, refrigerator surfaces, or onto bathroom surfaces, such as toilets, sinks, bathtubs, showers, and drains. Other suitable treatable surfaces are floor surfaces, door surfaces and window surfaces.

In one embodiment, the article is a component of process equipment, such as cooling equipment, water treatment equipment, air treatment equipment or food processing equipment. In one such embodiment, the component is selected from: a cooling tower, a water treatment plant, a dairy processing plant, a food processing plant, a chemical process plant, and a pharmaceutical process plant. For example, the article may be a filter for a water treatment system and/or an air treatment system. The article may be an item of agricultural equipment.

In one embodiment, the article is a toilet bowl, a sink, a bathtub, a drain, a high-chair, a work surface, a food processing machine, a food preparation area, an air handling unit (or component thereof), an air duct, an air filter, or an air conditioning unit.

The article may include the framework in an amount of 0.001 wt % or more, such as 0.01 wt % or more, or 0.1 wt % or more, such as 1 wt % or more, or 5 wt % or more. For example, the composition may include the framework in an amount of 80 wt % or less, such as 50 wt % or less, or 20 wt % or less. The composition may include the framework in an amount of from 0.001 wt % to 80 wt %, such as from 5 wt % to 50 wt %.

### Compositions

A composition comprises a framework of the present invention and a carrier. The composition may, for example, be for 3D printing (e.g. fused deposition modelling (FDM) printing). As such, the composition may be a 3D-printable ink. The composition may be a solid, a liquid or a gel.

The carrier may include, or be, a solvent, for example an organic solvent. The solvent may be selected from the list consisting of acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxy-ethane (glyme, DME), dimethyl-formamide (DMF), dimethyl sulfoxide (DMSO), 1,4-dioxane, ethanol, ethyl acetate, ethylene glycol, glycerol, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, tetrahydrofuran (THF), toluene, triethyl amine, o-xylene, m-xylene and p-xylene. Preferably the solvent is selected from the list consisting of NMP, methanol, nitromethane, acetonitrile, ethylene glycol, DMF, glycerol, and DMSO.

The carrier may include, or be, a polymer, such as acrylonitrile butadiene styrene (ABS), polylactic acid, acrylonitrile styrene acrylate (ASA), polyethylene terephthalate (PET), glycolized polyester, polycarbonate, polyether ether ketone (PEEK), polyetherketoneketone (PEKK), polyetherimide (e.g. ULTEM), polypropylene, polyamide (e.g. Nylon), polyvinyl alcohol, butene-diol vinyl alcohol, thermoplastic elastomer (TPE) or thermoplastic polyurethane (TPU). The polymer may be silicone (polysiloxane, e.g. silicone rubber) and/or polyurethane.

The carrier may include a polymer and a solvent, such as an organic solvent.

The composition may include the framework in an amount of 0.001 wt % or more, or 0.01 wt % or more, such as 0.1 wt % or more, such as 1 wt % or more, or 5 wt % or more. For example, the composition may include the framework in an amount of 80 wt % or less, such as 50 wt % or less, or 20 wt % or less. The composition may include the framework in an amount of from 0.001 wt % to 80 wt %, such as from 5 wt % to 50 wt %.

The composition may, for example, be a paint, varnish or sealant.

### Preferred Embodiments

-L-(R²-Lₒ)ₙ- may be represented by the following formula:

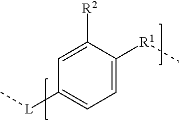

for example, wherein L is NH, R¹ is NH and/or S, R² is H and/or COOH. Such a group may repeat 1 or 2 or 3 or more times.

The framework (e.g. the linker) may comprise a group with the following formula:

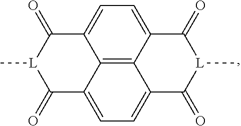

for example wherein L is N.

-L-(R²-Lₒ)ₙ- may be represented by one of the following formulae:

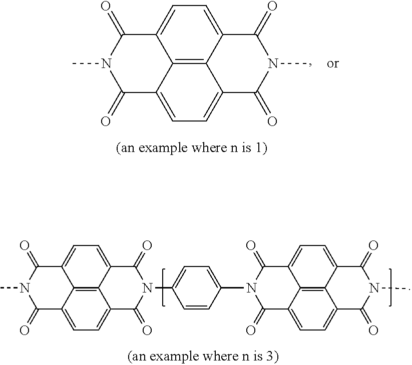

Such groups may repeat 1 or 2 or 3 or more times.

The framework (e.g. the linker) may comprise a group with the following formula:

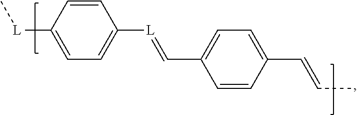

for example wherein L is NH. Such a group may repeat 1 or 2 or 3 or more times.

-L-(R²-Lₒ)ₙ- may be represented by one of the following formulae:

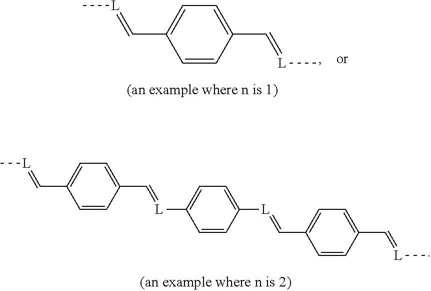

In one embodiment:
Each X bonds to three R¹ groups,
Each o is 1,
Each X group independently represents N, P, C1-3 alkyl, or a C3-10 (hetero)aryl group that is optionally substituted,
Each R¹ independently represents a C3-8 (hetero)aryl group,
Each L independently represents a group selected from the list consisting of: —CH₂—, —(CH₂)₂—, —OCH₂—, —SCH₂—, —N═CH—, —NHCH₂—, —O—, —S—, —SO₂—, —S(O)—, —NH—, —C(O)—, —C(O)O—, —C(O)NH—, —N(C(O)—)₂, —NHC(O)NH—, —NHC(O)O—, and —NHS(O)₂—,
Each R² independently represents a C3-16 (hetero)aryl group that is optionally substituted, and
The shortest path is on average 20 or more atoms in length.

In one embodiment:
Each X bonds to three R¹ groups,
Each o is 1,
Each X group independently represents N or a C3-8 (hetero)aryl group,
Each R¹ group independently represents a C3-10 aryl group,
Each L independently represents a group selected from the list consisting of: —OCH₂—, —SCH₂—, —N═CH—, —NHCH₂—, —O—, —S—, —NH—, —C(O)O—, —C(O)NH—, —N(C(O)—)₂, —NHC(O)NH—, and —NHC(O)O—,
Each R² may independently represent a C3-18 aryl group that is optionally substituted, and
The shortest path is on average 24 or more atoms.

In one embodiment:
Each X bonds to three R¹ groups,
Each o is 1,
Each X group independently represents N or pyridine, pyrimidine, triazine (e.g. 1,3,5-triazine), pyridazine and pyrazine,
Each R¹ group independently represents a C5-6 aryl group,
Each L independently represents a group selected from the list consisting of: —OCH₂—, —SCH₂—, —N═CH—, —NHCH₂—, —O—, —S—, —NH—, —C(O)NH—, —N(C(O)—)₂, and —NHC(O)NH—,
Each R² independently represents a C3-10 aryl group that is optionally substituted by one or more Y group independently selected from the list consisting of: cyano, halogen (e.g. F, Cl, Br and I), N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —OP(O)₂OR^{Z}, —S(O)₂NHR^{Z}, —NHS(O)₂R^{Z}, —NR^{Z}₂, —NHR^{Z} and —OR^{Z}, wherein any R^{Z} group independently represents H, C1-8 alkyl, C2-8 alkenyl, C6-8 aryl, or C4-8 heterocyclyl, and
The average shortest path is from 19 to 500 atoms in length.

In one embodiment:
Each X bonds to three R¹ groups,
Each o is 1,
Each X group independently represents N or triazine (e.g. 1,3,5-triazine),
R¹ represents a 6-membered (hetero)aryl ring, for example a phenyl group,
Each L independently represents a group selected from the list consisting of: —N═CH—, —S—, —NH—, and —N(C(O)—)₂,
Each R² may independently be selected from phenyl or napthyl that is optionally substituted with a group selected from the list consisting of: halogen (e.g. F and Cl), —C(O)OR^{Z}, —OC(O)R^{Z}, —C(O)NHR^{Z}, —NHC(O)R^{Z} and —OR^{Z}, wherein any R^{Z} group independently represents H, C1-8 alkyl, C2-8 alkenyl, C6-8 aryl, or C4-8 heterocyclyl,
The average shortest path is from 24 to 400 atoms,
90% or more of the linker units are attached to two or more (e.g. two) hub units, and
80% or more of the hub units are attached to three or more (e.g. three) linker units.

The framework may be represented by one of the following formulae:

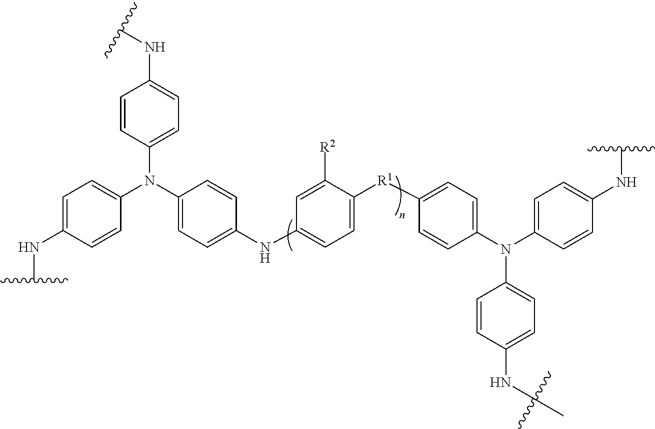

Wherein R¹ is NH and/or S; R² is H and/or COOH, and n is 1 or more, such as from 1 to 100, for example from 1 to 50, or from 2 to 100, for example from 2 to 50, or from 3 to 100, for example from 3 to 50. Preferably n is 2 or more.

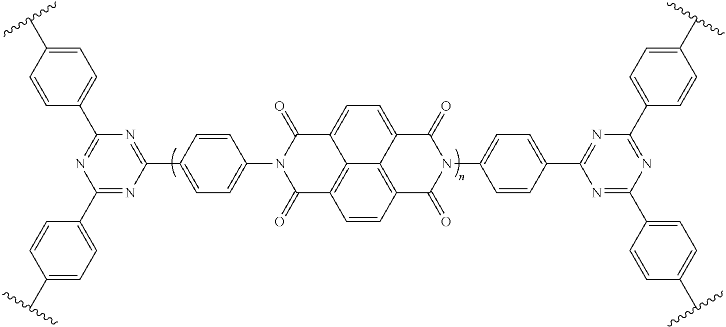

Wherein n is 1 or more, such as from 1 to 100, or from 2 to 100, for example from 2 to 50, or from 3 to 100, for example from 3 to 50. Preferably n is 2 or more.

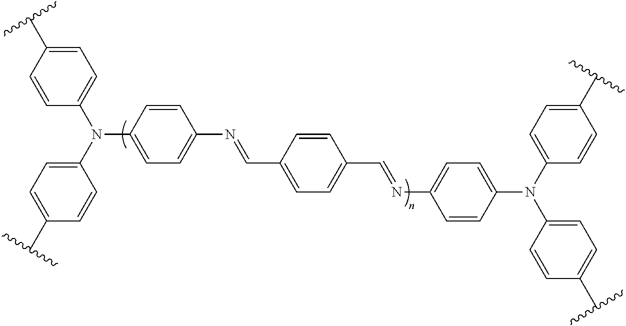

Wherein n is 1 or more, such as from 1 to 100, or from 2 to 100, for example from 2 to 50, or from 3 to 100, for example from 3 to 50. Preferably n is 2 or more.

### Examples

Preparation of Porous Organic Materials (POMs, i.e. Frameworks)

POMs were prepared by the conditions detailed below. In each case the POMs were subjected to Soxhlet extraction after synthesis in chloroform, methanol, and water (c.a. 500 mL each) for at least 12 hours to remove any trace starting materials.

To form POMs with absorbed antimicrobial materials (termed “POM/abs”) the POMs were dispersed in aqueous solutions of the antimicrobial materials via sonication for 10 mins, then stirred for 5 hours. The POM/abs materials were then isolated via filtration and subjected to Soxhlet extraction in water for 24 hours to ensure any non-absorbed materials were removed. Each POM/abs was initially loaded with 1% wt of the absorbed compound with respect to the POM.

### PTPA-Based POMs

The following PTPA-based POMs were prepared:

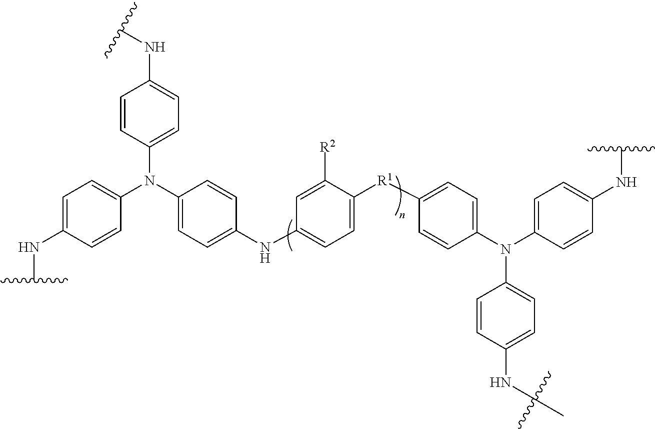

It will be understood that the wavy lines indicate bonds to further groups as shown in the parentheses and other triphenylamine groups, such that the structure shown repeats through the framework.

| POM name | R¹ | R² | n |
|---|---|---|---|
| PTPA | NH | H | 1 |
| PTPA 15 | NH | H | 3 |
| PTPA 50 | NH | H | 9 |
| PTPA S | S, NH* | H | 3 |
| PTPA COOH | NH | COOH | 1 |
| PTPA 15 COOH | NH | COOH and H | 3 |
| | | (copolymer) | |
| PTPA 200 | NH | H | 40 |

*The NH and S groups alternate.

It will be understood that the value of n is the average (mean) number of repeating groups. n can be reliably and repeatably controlled by adjusting the ratios of reagents used to prepare a framework. n may be determined using diffusion NMR.

A Schlenk tube was charged with tris(4-bromophenyl)amine (1 mmol), p-phenylenediamine and 1,4-dibromobenzene (1-153 mmol; it will be understood that increased amounts of these compounds increase the value of “n” and therefore provide longer linker lengths), Pd(dba)₂ (dba=dibenzylideneacetone, 17.3 mg, 0.03 mmol, 4 mol %), 2-dicyclohexylphosphino-2′,4′,6′-triisopropylbiphenyl (XPhos, 21.5 mg, 0.045 mmol), and sodium tert-butoxide (NaOtBu, 192.2 mg, 2 mmol) and placed under a nitrogen atmosphere.

Anhydrous toluene (50 mL) was added and the reaction mixture was heated under stirring to 110° C. After 48h, TLC analyses were carried out indicating complete consumption of the starting tris(4-bromophenyl)amine. The reaction was cooled to room temperature and solvents were then removed by centrifugation. The remaining solids were washed by chloroform, hot MQ water, and methanol (200 mL each), and then dried for 72 h in a vacuum oven to yield corresponding amine networks as black blue powders.

Replacement of 50 mol % of the 1,4-dibromobenzene with 2,5-dibromobenzoic acid provided PTPA COOH. Replacement of 1,4-diaminobenzene with 4,4′-diaminodiphenyl sulfide provided PTPA S.

### NPI and NPI 15

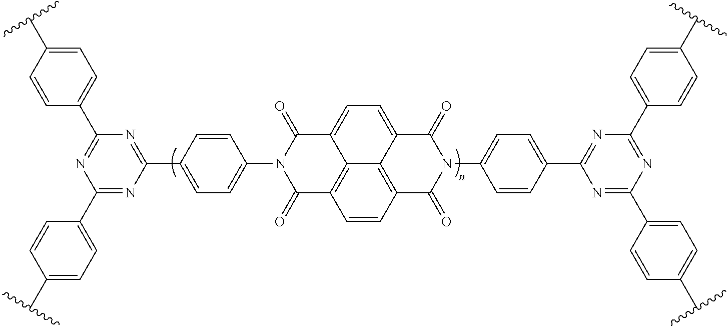

It will be understood that the wavy lines indicate bonds to further linker units as shown here and triphenyltriazine groups, such that the structure shown repeats through the framework.

| POM | |
|---|---|
| name | n |
| NPI 1 | 1 |
| NPI 15 | 3 |

A dried round-bottomed flask equipped with mechanical stirring, nitrogen inlet, Dean-Stark trap and a reflux condenser was charged with m-cresol (60 mL) and tris(4-aminophenyl)triazine (150 mg, 0.42 mmol). For NPI 15, the reaction vessel was also charged with phenylenediamine (45 mg, 0.42 mmol). After 5 min of stirring 1,4,5,8,-naphthalenetetracarboxylic dianhydride (457 mg, 1.69 mmol) was added and the reaction mixture stirred at room temperature for 30 min. The temperature was raised gradually to 180° C. and held for 72 hours. After cooling to 70° C., MeOH (50 mL) was added and the precipitate was collected and washed with additional DMF and methanol, water and acetone (3×50 mL each). The resulting product was dried at 80° C. under vacuum for 24h.

### Schiff Base 1 and Schiff Base 15

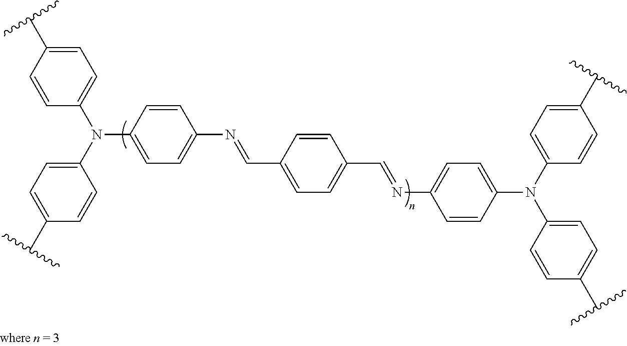

### Schiff Base 15

It will be understood that the wavy lines indicate bonds to further groups as shown in the parentheses and triphenylamine groups, such that the structure shown repeats through the framework.

A round bottomed flask was charged with tris(4-aminophenyl)amine (1 mmol), terephthalaldehyde (6 mmol), 1,4-diaminobenzene (3 mmol) and anhydrous DMSO (20 mL) and stirred at 160° C. for 4 hours under Dean-Stark conditions. Subsequently, crosslinked polymer was collected, washed with chloroform, hot MQ water and methanol (200 mL each), and then dried for 72 h in a vacuum oven to yield Schiff Base 15 as a red powder.

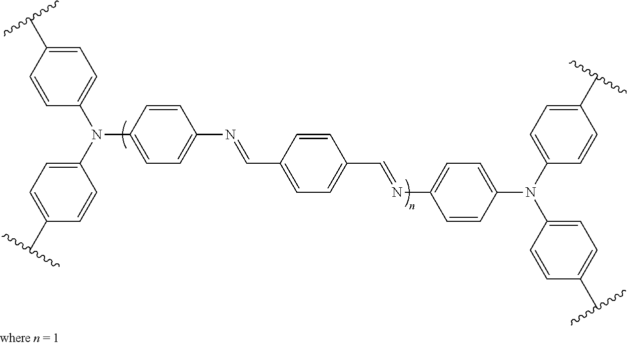

### Schiff Base 1

It will be understood that the wavy lines indicate bonds to further groups as shown in the parentheses and triphenylamine groups, such that the structure shown repeats through the framework.

To prepare Schiff Base 1, the procedure for Schiff Base 15 described above was followed except that no 1,4-diaminobenzene was added, and except that tris(4-aminophenyl)amine (1 mmol) and terephthalaldehyde (3 mmol) was used.

Absorbed Antibacterial Agents Chromium(III) was absorbed onto PTPA 15 from an aqueous solution to provide PTPA 15 Cr. Methylene blue was absorbed onto PTPA COOH 15 to provide PTPA COOH 15 MB. Amoxicillin was absorbed onto PTPA 15 from an aqueous solution, to provide PTPA 15 amox abs. Chromium(III), methylene blue and amoxicillin are known antibacterial agents.

In each case PTPA 15 or PTPA COOH 15 (100 mg) was sonicated in an aqueous solution of either Cr₂O₃, methylene blue or amoxicillin (each at 1 mg/mL, 5 mL) for 15 min. The resultant suspension was then allowed to stand for 24 hr, filtered and the resultant powder subjected to Soxhlet extraction in water for 48 hr.

Absorption of both the methylene blue and the chromium was determined by UV-vis absorption revealing no starting material left in the aqueous solution (i.e. complete absorption). For amoxicillin ¹H NMR analysis of a D20 study (with equivalent conditions) revealed complete absorption of the amoxicillin.

### PTPA 15 Amox

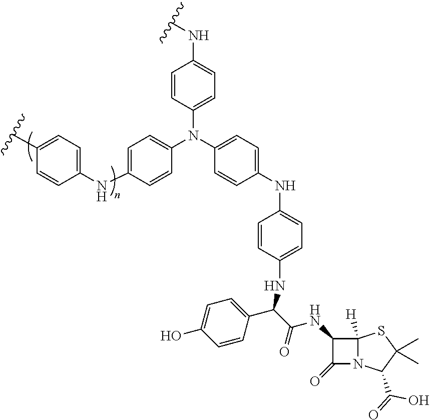

Where n=3 and the wavy lines of the aminophenyl linker unit attach to another amino-4-triphenylamino group, such that the structure shown repeats through the framework.

The same reaction conditions as used to prepare PTPA-15 were undertaken but with 1 wt % (with respect to diamine and tribromide starting materials) amoxicillin added to the reaction at start.

Infra-red analysis was used before and after Soxhlet extraction to confirm that the amoxicillin had covalently bonded to the PTPA 15 and was not simply absorbed onto the PTPA 15.

### Qualitative Antibacterial Activity

### Methodology

Two bacterial strains were selected to test the antimicrobial activity of the formed POMs and POM/abs: Moraxella catarrhalis (MX, gram negative) and Staphylococcus aureus (SA, gram positive). Both bacteria were propagated in saline conditions to give a suspension of 0.1 optical density at 625 nm, then 100 L or 50 L of each suspension were applied to HBHI and Mueller Hinton agar plates (for MX and SA respectively). To these plates c.a. 10 mg of POMs and POM/abs were placed on top of the bacterial lawns. The plates were incubated in the dark at 37° C. for 24 hours before being examined visually for any bactericidal exclusion zone around the POMs or POM/abs, or for bacterial growth up to/around the POMs or POM/abs.

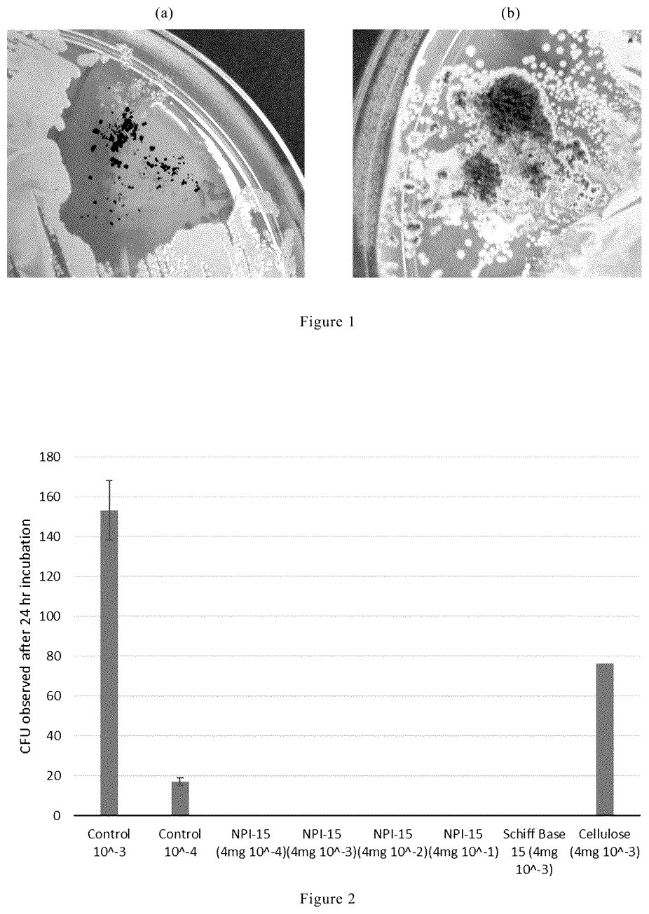

FIG. 1 of the accompanying drawings shows (a) a plate that has a bacterial exclusion zone around the POM, indicating antibacterial activity and (b) a plate that has bacterial growth up to and around the POM, indicating minimal or no antibacterial activity.

### Linker Length

Initial investigations were undertaken on lawns of SA to ascertain any efficacy in the formed POMs.

The following results were obtained for PTPA-based POMs:

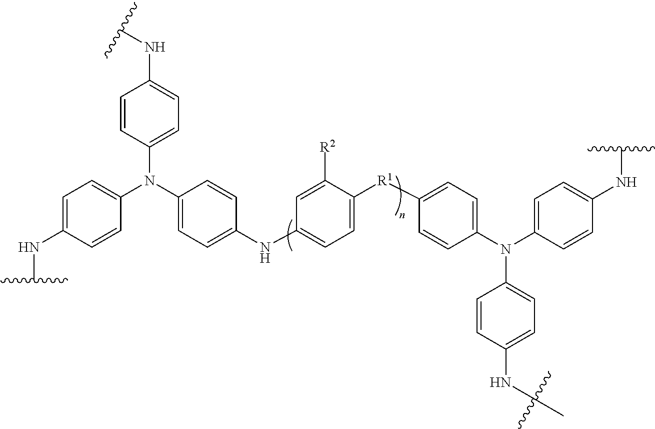

| | | | | Antibacterial activity | |
|---|---|---|---|---|---|
| | | | | observed? | |
| POM name | R¹ | R² | n | SA | MX |
| PTPA | NH | H | 1 | No | No |
| PTPA 15 | NH | H | 3 | Yes | No |
| PTPA 50 | NH | H | 9 | Yes | No |
| PTPA 200 | NH | H | 40 | Yes | No |
| PTPA S | S, NH | H | 1 | No | No |
| PTPA COOH | NH | COOH | 1 | No | No |
| PTPA 15 COOH | NH | COOH and H | 3 | Yes | No |
| | | (copolymer) | | | |

POMs synthesised using imide forming condensation reactions were also investigated:

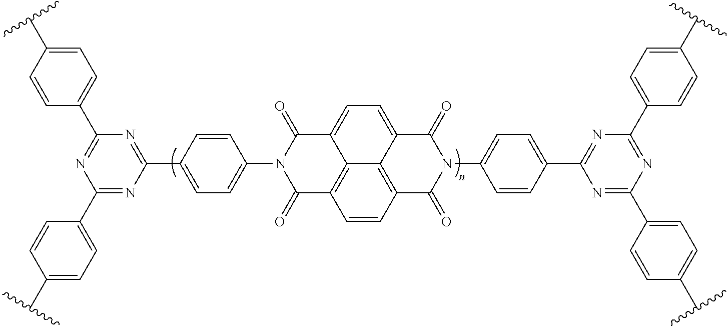

Antibacterial activity
observed?

| POM name | n | SA | MX |
|---|---|---|---|
| NPI 1 | 1 | No | No |
| NPI 15 | 3 | No | Yes |

The following results were obtained for Schiff base-based POMs:
Schiff Base 1 did not exhibit antibacterial activity against SA or MX.
Schiff Base 15 showed antibacterial activity against MX but was not active against SA.

Overall, it was observed that all frameworks having a longer linker length, and therefore having a larger pore size distribution, exhibited antibacterial activity.

### POM/abs

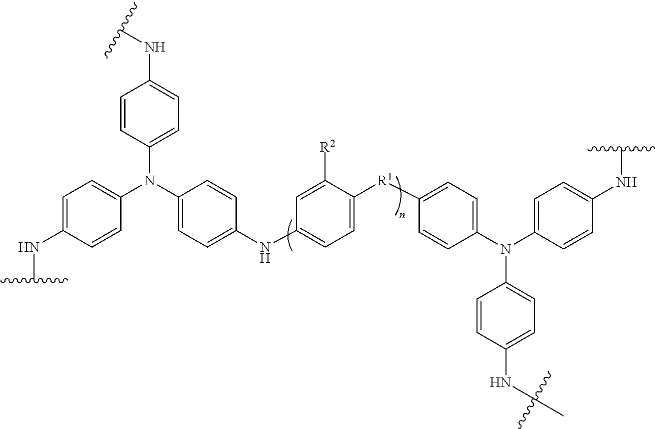

| | | | | Antibacterial activity | |
|---|---|---|---|---|---|
| | | | | observed? | |
| POM name | R¹ | R² | n | SA | MX |
| PTPA 15 Cr | NH | H | 3 | Not | Yes |
| | | | | tested | |
| PTPA 15 | NH | COOH and H | 3 | Not | Yes |
| COOH MB | | (copolymer) | | tested | |
| PTPA 15 | NH | H | 3 | Yes | Yes |
| amox abs | | | | | |

Both PTPA 15 Cr and PTPA 15 COOH MB exhibited antibacterial activity against MX. These POM/abs were not tested against SA.

It was found that PTPA 15 amox abs exhibited antibacterial activity against both SA and MX.

This shows that these frameworks can be used to absorb and retain antibacterial agents, and that the antibacterial agents retain their activity whilst they are absorbed into the POMs.

Therefore, POMs with absorbed antibacterial agents provide the surprising dual benefit of retaining the antibacterial activity of the antibacterial agent whilst also retaining the antibacterial agent within the structure of the POM.

### Bonded Antibacterial Agents

PTPA 15 amox demonstrated antibacterial activity against SA and MX.

This shows that antibacterial agents, such as amoxicillin, can be covalently bonded to POMs according to the invention to provide antibacterial materials.

### Comparative POMs

Other frameworks of intrinsic porosity, including PIMs (shown below), carbon black and cellulose also did not demonstrate any exclusion zones. Thus, porosity itself is not enough to give antimicrobial properties.

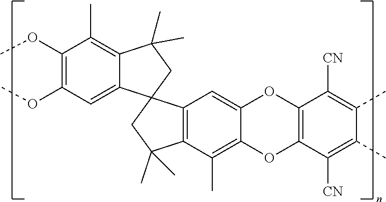

### Quantitative Antibacterial Activity

Antimicrobial efficacy was quantified by incubating bacterial solutions with the porous organic materials (POMs).

### Methodology

This methodology is based on an adapted method of the ‘ASTM E3160-18 Standard Test Method for Quantitative Evaluation of the Antibacterial Properties of Porous Antibacterial Treated Articles’.

Samples of POMs (either 4 mg or 1 mg±0.5 mg) were incubated with a suspension of Moraxella catarrhalis (of differing concentrations with respect to OD₆₀₀) in phosphate buffer solution (1 mL) at 37° C. for 24 hrs. The resulting settled suspension was decanted (50 L of the supernatant) onto either Muller Hinton or HPHI plates for 37° C. for 24 hrs and a colony count undertaken to determine bacterial death.

To determine the antibacterial efficacy with respect to Moraxella catarrhalis, the POMs NPI 15 and Schiff Base 15 were selected. Cellulose was selected as an organic porous material for a control. Moraxella catarrhalis was prepared at 0.1 OD₆₀₀ and diluted to 10⁻², 10⁻³ and 10⁻⁴ in PBS (10⁻² dilutions showed too much activity to provide meaningful quantifiable data).

### Results

FIG. 2 of the accompanying drawings shows colony forming units (CFU) of Moraxella observed via plate count method.

Total bacterial death was observed in each of the solutions incubated with NPI 15 and Schiff Base 15. Control samples showed bacteria was still present.

For the cellulose control, only c.a. 50% bacteria growth was observed demonstrating that absorption of bacteria into this comparative porous material is not enough to achieve complete bacterial inhibition.

In the case of NPI 15 further tests to find the limits of bactericidal properties were undertaken with the POM incubated at both 0.1 OD₆₀₀ and the diluted to 10⁻² at 1 mg and 4 mg amounts.

In each case no colony forming units were observed after incubation. This demonstrates that the frameworks of the present invention can be active even at very low concentrations.

Antibiotic Effects when Blended into Article

To assess the potential of the antibacterial capabilities of the porous networks in blended networks, NPI-15 was taken as a case study, owing to its efficacy against Moraxella catarrhalis.

1% wt of NPI-15 was blended with the commercially available Ecoflex™ (addition-cure silicone rubber pre-polymerization). The mixture was allowed to cure overnight. The efficacy of the cured mixture against Moraxella catarrhalis (MX) was determined by incubation of each sample (0.2 g) in PBS:bacteria solutions for 24 hr at 37° C. A control sample contained the same bacteria with no exposure to NPI or Ecoflex. The samples were then plated onto HBHI growth media and, after a further 24 hr of growth at 37° C., colony forming units (CFUs) were counted (average of 4 runs reported).

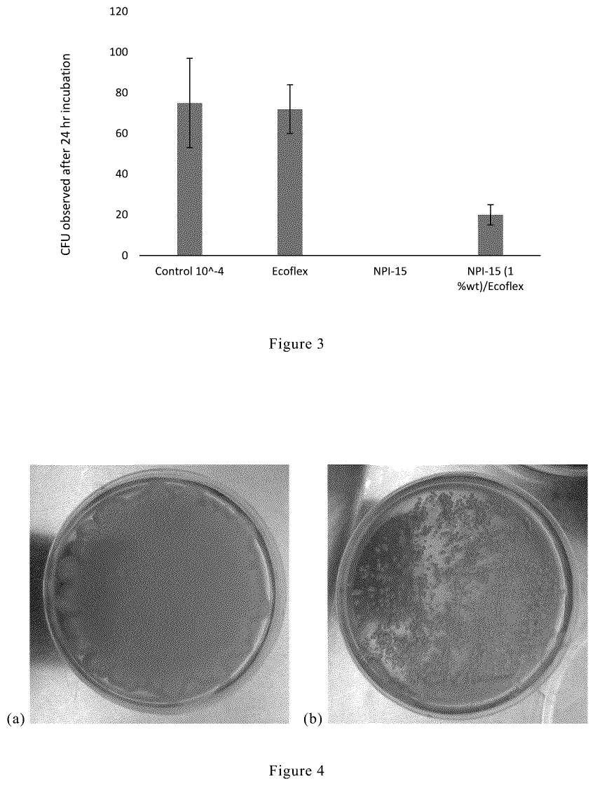

FIG. 3 of the accompanying drawings shows the CFU count after 24 hours of incubation of the bacteria with the control, Ecoflex alone, NPI-15 alone, or the combination of NPI-15 (1 wt %) in Ecoflex.

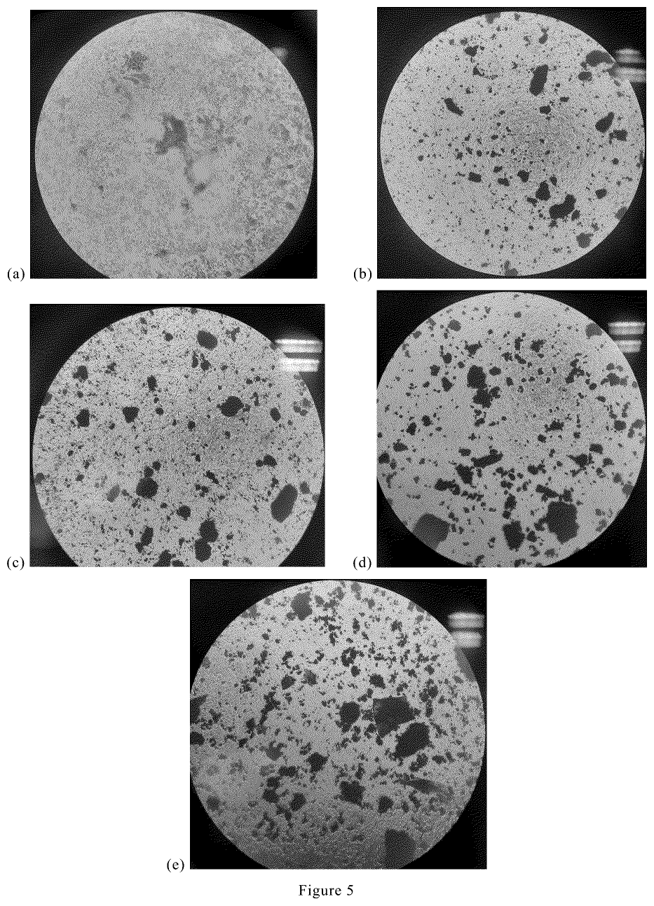

FIG. 4 of the accompanying drawings shows (a) a saturated lawn of MX bacteria (i.e. control), and (b) the same solution exposed to NPI-15 (1 wt %) combined with Ecoflex at 0.2 g for 24 h at 37° C.

Ecoflex alone did not display antibacterial properties, as with the control sample.

NPI-15 displayed significant antibacterial properties when alone and when combined with Ecoflex.

This shows that the NPI-15:Ecoflex blend still had desirable antibacterial properties when compared to pure Ecoflex or the control (grown bacteria with no contact with NPI or Ecoflex), as shown in FIGS. 1 and 2.

Therefore the antibacterial properties of the frameworks of the present invention still remain when the framework is blended with another material to form an article.

### Lack of Cytotoxicity

MTT assays were used to assess metabolic activity. Over a period of 7 days, NPI-15, Schiff Base 15, PTPA 15 and PTPA 15 COOH were subjected to cytotoxicity screening using qualitative visual scoring. Cell line: MRC-5 (ATCC® CCL-171™) - Population doubling: 30.2. Cell culture medium: Eagle's Minimum Essential Medium (EMEM); product test concentrations: ten-fold dilutions starting at 8%; contact time(s): 24 hours; incubation condicution: 37° C.±2° C. and 5% CO₂.

0.1% w/v Triton X-100 was used as a positive control. MRC-5 cells (2×10⁴ seeded per well) were used as a negative control.

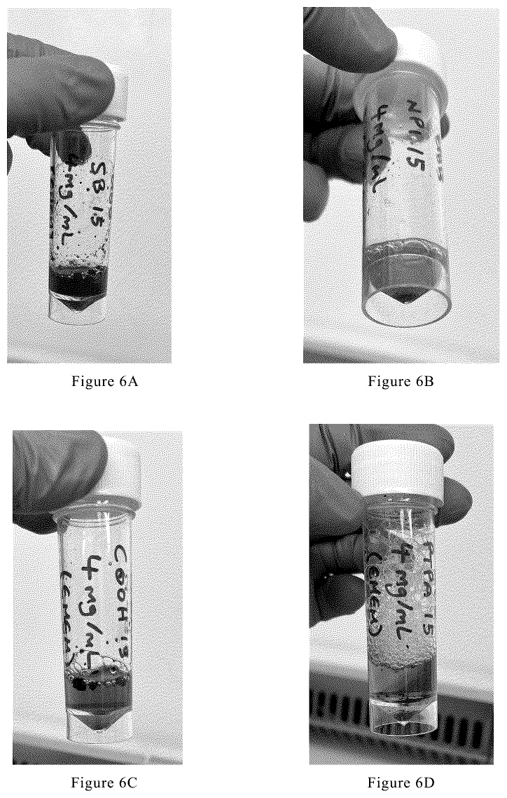

FIG. 6 of the accompanying drawings shows the four polymers (4 mg/mL) in the MTT assays with no colour change after 4 hours of contact with the live cells. Specifically, FIG. 6A shows Schiff Base 15, FIG. 6B shows NPI 15, FIG. 6C shows PTPA 15 COOH, and FIG. 6D shows PTPA 15.

In each case, the frameworks of the invention caused no cytotoxicity or reduction in cell growth. Purple colouring attributed to cell survival was retained in the presence of each framework.

Qualitative morphological grading of the cytotoxicity of each of the frameworks on MRC-5 cells, starting at 8% (the highest concentration cells are exposed to in suspension test) and serially diluted 10-fold, according to visual microscopy scoring criteria following 24 hours of treatment. Controls were used at a single concentration.

| | Concentration | | | | | | | |
|---|---|---|---|---|---|---|---|---|
| | | 8% | | | | | | |
| Sample | 100% | (10⁻¹) | 10⁻² | 10⁻³ | 10⁻⁴ | 10⁻⁵ | 10⁻⁶ | 10⁻⁷ |
| 0.1% w/v | 4 | n/a | n/a | n/a | n/a | n/a | n/a | n/a |
| Triton X-100 | | | | | | | | |
| (Positive | | | | | | | | |
| Control) | | | | | | | | |
| MRC-5 cells | 0 | n/a | n/a | n/a | n/a | n/a | n/a | n/a |
| (Negative | | | | | | | | |
| Control) | | | | | | | | |
| NPI 15 | n/a | 0 | 0 | 0 | 0 | 0 | 0 | 0 |
| COOH 15 | n/a | 0 | 0 | 0 | 0 | 0 | 0 | 0 |
| SB 15 | n/a | 0 | 0 | 0 | 0 | 0 | 0 | 0 |
| PTPA 15 | n/a | 0 | 0 | 0 | 0 | 0 | 0 | 0 |

This shows that, as well as displaying antibiotic activity, frameworks of the present invention are not cytotoxic to MRC-5 cells.

In further tests, samples were plated on serial dilutions onto mouse fibroblasts, in a Median Tissue Culture Infectious Dose (TCID50) assay. Images were taken after two hours of contact. In each case the images showed intact cells, showing that the frameworks were not cytotoxic to the fibroblasts.

FIGS. 5a-e of the accompanying drawings show the images taken after two hours of contact with a framework according to the invention. FIG. 5a shows the image for a control sample (no framework). FIG. 5b shows the image for the PTPA 15 framework at 10 μg/mL. FIG. 5c shows the image for the COOH 15 framework at 8 μg/mL. FIG. 5d shows the image for the Schiff Base 15 framework at 10 μg/mL. FIG. 5e shows the image for the NPI 15 framework at 10 μg/mL. Concentrations relate to the weight of the framework per mL of tissue culture.

This shows that, as well as displaying antibiotic activity, frameworks of the present invention are not cytotoxic to mouse fibroblasts.

1. A framework comprising hub units joined by linker units, wherein:
each hub unit comprises an X group bonded to three or more R¹ groups, wherein:
each X group independently represents N, P or a C1-10 hydrocarbon group that is optionally substituted;
each R¹ group independently represents a C3-10 (hetero)aryl group that is optionally substituted;
each linker unit links two or more hub units together and is represented by the formula -L-(R²-Lₒ)ₙ-, wherein:
each L group is independently selected from the list consisting of: —CH₂—, —(CH₂)₂—, —(CH)₂—, —CC—, —OCH₂—, —SCH₂—, —P═CH—, —PHCH₂—, —N═CH—, —NHCH₂—, —O—, —O₂—, —OS—, —S—, —SO₂—, —S(O)—, —S₂—, —N═N—, —ONH—, —NH—, —P═N—, —P(O)HNH—, —POOHNH—, —OPOOHNH—, —P(O)HO—, —POOHO—, —OPOOHO—, —P═P—, —OPH—, —PH—, —C(O)—, —C(O)O—, —C(O)NH—, —N(C(O)—)₂, —NHC(O)NH—, —NHC(O)O—, and —NHS(O)₂—;
each R² group independently represents a C3-20 (hetero)aryl group that is optionally substituted;
each o is 1 or more; and
each n is 1 or more; and
the R¹ groups of the hub unit are bonded to L groups of the linker units, such that three or more linker units are bonded to each hub unit; and
a) the shortest path from the most central atom of each hub unit to the most central atom of an adjacent hub unit, inclusive of the most central atoms, is on average 19 atoms or more, and/or
b) the framework is bonded to an antimicrobial agent, and/or
c) the framework is complexed with an antimicrobial agent.

2. (canceled)

3. (canceled)

4. The framework of claim 1, wherein each hub unit comprises an X group bonded to three R¹ groups.

5. The framework of claim 1, wherein each X group independently represents N or a C3-8 (hetero)aryl group.

6. The framework of claim 1, wherein each R¹ group independently represents a C3-10 aryl group.

7. The framework of claim 1, wherein each L independently represents a group selected from the list consisting of: —OCH₂—, —SCH₂—, —N═CH—, —NHCH₂—, —O—, —S—, —NH—, —C(O)O—, —C(O)NH—, —N(C(O)—)₂, —NHC(O)NH—, and —NHC(O)O—.

8. The framework of claim 1, wherein each R² independently represents a C3-18 aryl group that is optionally substituted.

9. The framework of claim 1, wherein the shortest path from the most central atom of each hub unit to the most central atom of an adjacent hub unit, inclusive of the most central atoms, is on average from 24 to 400 atoms.

10. (canceled)

11. (canceled)

12. (canceled)

13. The framework of claim 1, wherein n is 2 or more.

14. The framework of claim 1, wherein option a) applies, and wherein the framework is defined by one of the following formulae: 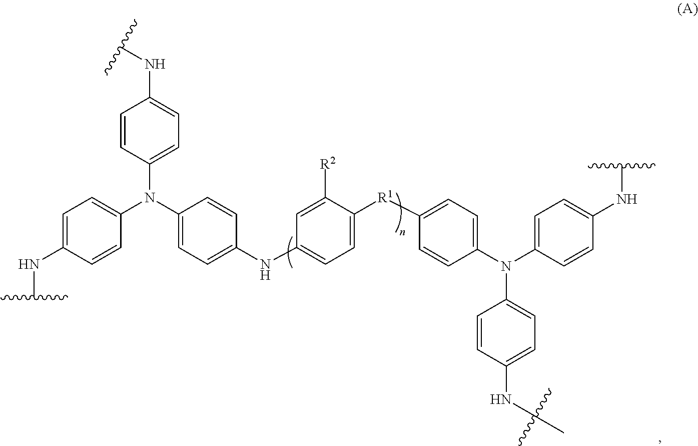
wherein: each R¹ is selected from NH and S, each R² is selected from H and COOH, and n is 2 or more; or 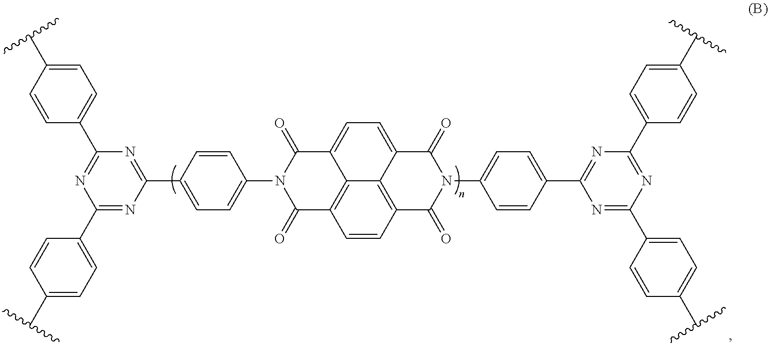
wherein n is 2 or more; or 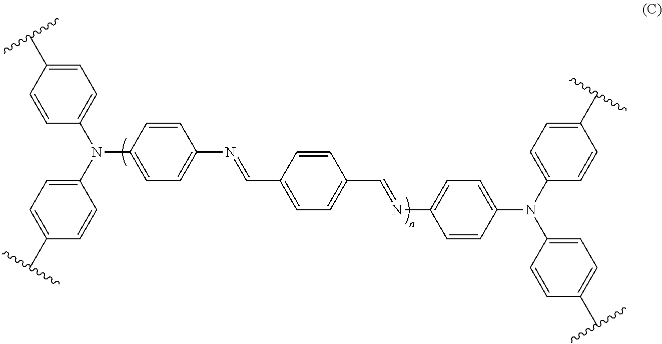
wherein n is 2 or more.

15. The framework of claim 1, wherein the antimicrobial agent is an antiviral agent, an antifungal agent and/or an antibacterial agent.

16. An article comprising a framework as defined by claim 1.

17. The article of claim 16, wherein the article is a medical device, or a part thereof.

18. The article of claim 17, wherein the medical device is a dressing, a surgical instrument, an implant, a prosthesis, or a medical machine.

19. The article of claim 16, wherein the article is a food preparation surface, a bathroom surface, a flooring surface, a door, a window, an item of water or air processing equipment, an item of agricultural equipment, or a part thereof.

20. The article of claim 16, wherein the framework forms at least the part of the article comprising the surface, or wherein the framework is included in a coating on the surface of the article.

21. A composition comprising a framework of claim 1 and a carrier.

22. The composition of claim 21, wherein the carrier includes an organic solvent.

23. The composition of claim 21, wherein the carrier includes a polymer.

24. The composition of claim 21, wherein the composition is a paint, varnish or sealant.
